(19) United States
(12) Patent Application Publication
(10) Pub. No.: US 2007/0037169 A1
(43) Pub. Date: Feb. 15, 2007

(54) Selective Dehybridization using Electrochemically-Generated Reagent on an Electrode Microarray

(75) Inventors: John J. Cooper Jr., Seattle, WA (US); Andrei Gindilis, Mukilteo, WA (US)

Correspondence Address: COMBIMATRIX CORPORATION, 6500 HARBOUR HEIGHTS PARKWAY, MUKILTEO, WA, 98275, US

(73) Assignee: COMBIMATRIX CORPORATION, Mukilteo, WA (US)

(21) Appl. No.: 11/161,607

(22) Filed: Aug. 9, 2005

(51) Int. Cl. C12Q 1/68

(52) U.S. Cl. 435/6; 204/450

(57) ABSTRACT

The present invention provides a method for selective dehybridization by electrochemically-generated (ECG) reagent on an electrode microarray. The ECG reagent is generated by activation of selected electrodes. Activation alters pH in the vicinity of only the selected electrodes. In one embodiment, the increase or decrease in pH is sufficient to cause dehybridization of an oligonucleotide duplex at the selected electrodes. In another embodiment, the increase or decrease in pH is sufficient to prevent chemical dehybridization at the selected electrodes. The dehybridized single stranded target oligonucleotide may be recovered and amplified by PCR.

### TECHNICAL FIELD OF INVENTION

This invention provides a selective oligonucleotide dehybridization method that uses electrochemically-generated reagents to effect dehybridization on a selective or localized basis. Preferably, the inventive process is performed on an electrode microarray. Specifically, this invention provides electrochemical generation of acidic or basic reagents at one or more active electrodes, wherein the electrochemically-generated reagents alter pH sufficiently (a) to cause dehybridization of an oligonucleotide duplex at the active electrode or (b) to prevent chemically induced dehybridization of an oligonucleotide duplex at the active electrode.

### BACKGROUND OF THE INVENTION

Microarrays have become important analytical research tools in pharmacological and biochemical research and discovery. Microarrays are miniaturized arrays of points on a solid surface. Molecules, including biomolecules, may be attached or synthesized in situ at specific attachment points on a microarray. The attachment points are usually in a column and row format although other formats may be used. An advantage of microarrays is that they provide the ability to conduct hundreds, if not thousands, of experiments in parallel. Such parallelism, as compared to sequential experimentation, can be used to increase the efficiency of exploring relationships between molecular structure and biological function, where slight variations in chemical structure can have profound biochemical effects.

Microarrays are available in different formats and have different surface chemistry characteristics. The differences result in different approaches for attaching or synthesizing molecules on a microarray. Differences in surface chemistry lead to differences in preparation methods for providing a surface that is receptive to attachment of a pre-synthesized chemical species or for synthesizing a chemical species in situ.

Research using microarrays has focused mainly on deoxyribonucleic acid (DNA) and ribonucleic acid (RNA) related areas, which includes genomics, cellular gene expression, single nucleotide polymorphisms (SNP), genomic DNA detection and validation, functional genomics, and proteomics (Wilgenbus and Lichter, J. Mol. Med. 77:761, 1999; Ashfari et al., Cancer Res. 59:4759, 1999; Kurian et al., J. Pathol. 1 87:267, 1999; Hacia, Nature Genetics 21 suppl.:42, 1999; Hacia et al., Mol. Psychiatry 3:483, 1998; and Johnson, Curr. Biol. 26:R171, 1998.)

There are numerous methods for preparing a microarray of DNA related molecules. DNA related molecules include native or cloned DNA and synthetic DNA. Synthetic, relatively short single-stranded DNA or RNA strands are commonly referred to as oligonucleotides. Microarray preparation methods include the following: (1) spotting a solution on a prepared flat surface using spotting robots; (2) in situ synthesis by printing reagents via ink jet or other printing technology and using regular phosphoramidite chemistry; (3) in situ parallel synthesis using electrochemically-generated acid for deprotection and using regular phosphoramidite chemistry; (4) maskless photo-generated acid (PGA) controlled in situ synthesis and using regular phosphoramidite chemistry; (5) mask-directed in situ parallel synthesis using photo-cleavage of photolabile protecting groups (PLPG); (6) maskless in situ parallel synthesis using PLPG and digital photolithography; and (7) electric field attraction/repulsion for depositing oligonucleotides.

Photolithographic techniques for in situ oligonucleotide synthesis are disclosed in Fodor et al. U.S. Pat. No. 5,445,934 and the additional patents claiming priority thereto. Electric field attraction/repulsion microarrays are disclosed in Hollis et al. U.S. Pat. No. 5,653,939 and Heller et al. U.S. Pat. No. 5,929,208. An electrode microarray for in situ oligonucleotide synthesis using electrochemical deblocking is disclosed in Montgomery, U.S. Pat. Nos. 6,093,302, 6,280,595, and 6,444,111 (Montgomery I, II, and III respectively), which are incorporated by reference herein. Parallel rows of linear electrodes for in situ oligonucleotide synthesis at adjacent surfaces, but not on the array, is disclosed in Southern, U.S. Pat. No. 5,667,667. A review of oligonucleotide microarray synthesis is provided by: Gao et al., Biopolymers 73:579, 2004.

The electrochemical synthesis microarrays disclosed in Montgomery I, II, and III is based upon a semiconductor chip having a plurality of microelectrodes. In order to provide appropriate reactive groups at each electrode, the microarray is coated with a porous matrix material. The electrodes are “turned on” by applying a voltage or current that generates electrochemical reagents that alter the pH in a small, defined “virtual flask” region or volume adjacent to the electrode. The electrochemically-generated reagents remove acid-labile or base labile protective groups to allow continued synthesis of a DNA or other oligomeric or polymeric material. The pH changes only in the vicinity of the electrode because the ability of the acidic or basic reagent to travel away from an electrode is limited by natural diffusion and by buffering.

Isolation and separation of a particular single stranded DNA or RNA is useful in studies related to genomics, cellular gene expression, single nucleotide polymorphisms, genomic DNA detection and validation, functional genomics, and proteomics. Microarrays can be advantageously used in such studies. Research related to DNA and RNA can benefit from better isolation and separation of the genetic strands of interest from unwanted sequences. However, separation and purification methods are generally cumbersome and time consuming.

A characteristic of DNA is that complementary single DNA strands will hybridize to form a double-stranded duplex. Additionally, complementary single strands of ribonucleic acid (RNA) may also hybridize to form a duplex. Moreover, complementary single strands of DNA and RNA may hybridize to form a hybrid DNA-RNA nucleic acid duplex.

Another characteristic of nucleic acid duplexes is that they can be dehybridized, which provides the single strands of nucleic acid molecules free from association. Known variables that affect dehybridization include temperature, time of treatment, pH, helicase enzymes, binding proteins, hydrogen bonding disruptors, and buffer/electrolyte.

Whether hybridizing single strands of nucleic acid or dehybridizing nucleic acid duplexes, the available methods generally do not allow any selectivity within a particular bulk solution of nucleic acids. Thus, all nucleic acid strands in a particular solution are subjected to the same conditions of temperature, pH, salt, etc. For example, heat dehybridization of a bulk solution causes all duplexes having a melting temperature below the solution temperature to dehybridize. Thus, while many dehybridization techniques are known for bulk dehybridization, there is no known technique to selectively dehybridize different duplexes as all are exposed to the same conditions. In order to isolate a particular single-stranded nucleic acid sequence from a bulk solution of nucleic acid duplexes having the same melting temperature, an additional separation method is required other than dehybridization in a bulk solution.

Bulk dehybridization methods have inherent problems. For example, heating nucleic acid duplexes to high temperature to cause dehybridization can cause damage to the duplexes, especially if one of the strands of a duplex is also attached to a solid surface as in a microarray device. As another example, the use of alkali requires addition of acid to neutralize the alkali prior to further use of single stranded nucleic acid such as in hybridization studies for gene expression. Furthermore, any additive such as helicase enzymes, binding proteins, hydrogen bonding disruptors, or buffer may interfere with further use or processing of single stranded nucleic acid material. Additional cleaning steps may be required to remove such additives.

U.S. Pat. Nos. 6,613,527, 6,395,489, 6,365,400, and 6,197,508 and U.S. Pat. Nos. 5,824,477, 5,527,670, 6,291,185 and 6,033,850 provide a method for dehybridizing (denaturing) native double-stranded nucleic acid material using an electric field and methyl viologen dichloride. A voltage applied to electrodes (within an ionic buffer solution) generates the electric field. The electric field directly causes denaturation when the double-stranded nucleic acid material is within an electric field. This method is a bulk dehybridization method using an electric field that operates on the entire nucleotide solution rather than on selected oligonucleotide duplexes because there is no means of confinement. Thus, this method does not allow selective dehybridization because all duplexes within an unconfined electric field will dehybridize regardless of the sequence.

U.S. Pat. Nos. 6,824,740 and 6,129,828 disclose a prophetic sample purification device having a denaturation region. The denaturation region is a bulk denaturation without selectivity. The method uses heat on a bulk solution to dehybridize the oligonucleotide duplexes and thus is not selective to any particular oligonucleotide duplex.

U.S. Pat. Nos. 6,245,508, 6,048,690, and 5,849,486 disclose the use of an electric field on an electronic microarray test pad for partial dehybridization of a fluorescently labeled target from a pre-attached biotinylated capture probe. This method of dehybridization/denaturation does not disclose selective dehybridization based upon a selected sequence because the probes are pre-selected to be complementary to the targets and are pre-synthesized and attached to the test pads.

U.S. Patent Appl. Pub. No. U.S. 2005/0009020 (Distler) provides a method of dehybridization of oligonucleotides on a DNA array by immersion in hot water (75° C.) This method relies upon the use of hot water and thus damage to the DNA may result.

U.S. Pat. No. 5,849,486 discloses the prophetic use of a restriction enzyme to remove selected sequences from a hybridized oligonucleotide. This method does not provide for selective dehybridization of a hybridized oligonucleotide.

U.K. Pat. Appl. Pub. No. 2,247,889 discloses non-selective denaturation of DNA into its single-stranded form in an electrochemical cell. The theory is stated to be that there is electron transfer to the DNA at the interface of an electrode, which effectively weakens the double-stranded structure and results in separation of the strands. This method does not disclose selective dehybridization because any and all nucleotide duplexes are subjected to the effects of the electrochemical cell.

As is apparent from, the prior art on dehybridization of oligonucleotide duplexes, generally uses conventional methods (such as heat or raising pH or an electric field) to dehybridize duplexes in a bulk solution or on a test pad in a non-selective manner. Thus, there is a need in the art to provide for a method to selectively dehybridize a nucleic acid duplex. Therefore, there is a need in the art for a selective dehybridization method based upon a particular sequence to provide a means for better separation and isolation of oligonucleotides for further processing.

### SUMMARY OF THE INVENTION

The present invention provides a method for selective dehybridization by confined electrochemically-generated reagents on an electrode microarray for selective recovery of a single type of hybridized target oligonucleotide, hybridized to a probe oligonucleotide. The method provides an electrode microarray device having a plurality of electrodes on a top or first surface of the device, wherein the plurality of electrodes is proximate to a porous reaction layer. The porous reaction layer contains at least one oligonucleotide duplex. The oligonucleotide duplex comprises a target oligonucleotide hybridized to a probe oligonucleotide. A dehybridizing solution bathes the porous reaction layer and the surfaces of the electrodes. One or more electrodes are activated to generate electrochemically-generated reagents, wherein the reagents generated dehybridize the duplex located within the porous reaction layer adjacent to an activated electrode. The target oligonucleotide is dehybridized (that is, becomes unattached) and goes into the dehybridizing solution. The probe oligonucleotide substantially remains attached within the porous reaction layer.

Preferably, the porous reaction layer is attached to the at least one electrode. Alternatively, the porous reaction layer is attached to an opposing surface to the electrode surface. Preferably, the probe oligonucleotide is synthesized in situ by an electrochemical synthesis means. Preferably, the electrochemical synthesis means comprises a phosphoramidite synthesis means and an electrochemical deblocking means. Alternatively, the probe oligonucleotide is presynthesized and attached at known locations within the porous reaction layer.

Preferably, the activation means comprises application of a constant current to the at least one electrode of an absolute value of approximately 0.1 to 20 microampere per electrode. Alternatively, the activation means comprises application of a constant voltage to at least one electrode of an absolute value of approximately 0.1 to 10 volts. Preferably, the voltage is an absolute value of approximately 1 to 5 volts. Preferably, the electrochemically-generated reagent is a base. Alternatively, the electrochemically-generated reagent is an acid. Preferably, the dehybridization solution comprises a buffer having a concentration of approximately 1 to 1000 millimolar and a pH of approximately 5 to 9. The buffer is selected from the group consisting of di-sodium phosphate, mono-sodium phosphate, citrate, carbonate, bicarbonate, borate, acetate, MES, Bis-Tris, ADA, ACES, PIPES, MOPSO, Bis-Tris Propane, BES, MOPS, HEPES, TES, DIPSO, TAPSO, TRIZMA, HEPPSO, POPSO, EPPS, TEA, Tricine, Bicine, TAPS, AMPSO, CHES, CAPSO, AMP, CAPS and combinations thereof. Preferably, the probe oligonucleotide is DNA. Alternatively, the probe oligonucleotide is RNA. Preferably, the target oligonucleotide is DNA. Alternatively, the target oligonucleotide is RNA.

The present invention further provides a method for selective dehybridization by electrochemically-generated reagent on an electrode microarray for selective recovery of one or more target oligonucleotides. An electrode microarray is provided having an electrode surface. The surface has at least a first electrode and a second electrode. The first electrode is proximate to a first reaction porous layer having a plurality of first oligonucleotide duplexes. The second electrode is proximate to a second porous reaction layer having a plurality of second oligonucleotide duplexes, and so on. Each of the plurality of first oligonucleotide duplexes comprises a first probe oligonucleotide and a first target oligonucleotide. Each of the plurality of second oligonucleotide duplexes comprises a second probe oligonucleotide and a second target oligonucleotide, and so on. A dehybridizing solution contacts the first porous reaction layer, the second porous reaction layer and subsequent porous reaction layers, and each electrode surface. At least one of the plurality of first duplexes is dehybridized by an electrochemically-generated reagent. The electrochemically-generated reagent is generated by an activation means applied to the first electrode. The first target oligonucleotide dehybridizes into the dehybridization solution. The first probe oligonucleotide substantially remains attached to the first porous reaction layer. The plurality of second oligonucleotide duplexes remains hybridized and attached to the second porous reaction layer.

Preferably, the first porous reaction layer is attached to the first electrode and the second porous reaction layer is attached to the second electrode. Alternatively, the first porous reaction layer is attached to an opposing surface to the electrode surface and the second reaction layer is attached to an opposing surface to the electrode surface. Preferably, the first probe oligonucleotide and the second probe oligonucleotide are synthesized in situ by an electrochemical synthesis means. Preferably, the electrochemical synthesis means comprises a phosphoramidite synthesis means and an electrochemical deblocking means. Alternatively, the first probe oligonucleotide and the second probe oligonucleotide are presynthesized and attached to the reaction layer.

Preferably, the activation means comprises: application of a constant current to the first electrode of an absolute value of approximately 0.1 to 20 microampere per electrode. Alternatively, the activation means comprises application of a constant voltage to the at least one electrode of an absolute value of approximately 0.1 to 10 volts. Preferably, the voltage is an absolute value of approximately 1 to 5 volts. Preferably, the electrochemically-generated reagent is a base. Alternatively, the electrochemically-generated reagent is an acid. Preferably, the dehybridization solution comprises a buffer having a concentration of approximately 1 to 1000 millimolar and a pH of approximately 5 to 9. The buffer is selected from the group consisting of di-sodium phosphate, mono-sodium phosphate, citrate, carbonate, bicarbonate, borate, acetate, MES, Bis-Tris, ADA, ACES, PIPES, MOPSO, Bis-Tris Propane, BES, MOPS, HEPES, TES, DIPSO, TAPSO, TRIZMA, HEPPSO, POPSO, EPPS, TEA, Tricine, Bicine, TAPS, AMPSO, CHES, CAPSO, AMP, CAPS and combinations thereof. Preferably, the first probe oligonucleotide and the second probe oligonucleotide are DNA. Alternatively, the first probe oligonucleotide and the second probe oligonucleotide are RNA. Preferably, the first target oligonucleotide and the second target oligonucleotide are DNA. Alternatively, the first target oligonucleotide and the second target oligonucleotide are RNA.

The present invention further provides a method for selective dehybridization of hybridized nucleic acids, comprising electrochemically-generating a reagent at, at least one electrode on an electrode microarray device and recovering of a single sequence of target oligonucleotide. The electrode microarray has an electrode surface having at least one electrode proximate to a porous reaction layer. At least one probe oligonucleotide is bound to the porous reaction layer. A target oligonucleotide is hybridized to the at least one probe oligonucleotide. The target oligonucleotide and the probe oligonucleotide form an oligonucleotide duplex. An electrochemically-generated reagent dehybridizes the oligonucleotide duplex. The reagent is generated in a dehybridizing solution contacting the porous reaction layer and the electrode surface. The electrochemically-generated reagent is generated by an activation means applied to the at least one electrode. The target nucleotide goes into the dehybridizing solution. The probe oligonucleotide substantially remains attached to the reaction layer. The dehybridizing solution having the target nucleotide is recovered.

Preferably, the porous reaction layer is attached to the at least one electrode. Alternatively, the porous reaction layer is attached to an opposing surface to the electrode surface. Preferably, the probe oligonucleotide is synthesized in situ by an electrochemical synthesis means. Preferably, the electrochemical synthesis means comprises a phosphoramidite synthesis means and an electrochemical deblocking means. Alternatively, the probe oligonucleotide is presynthesized and attached to the reaction layer.

Preferably, generating an electrochemical reagent comprises applying a constant current to at least one electrode, wherein the constant current has an absolute value of approximately 0.1 to 20 microampere per electrode. Alternatively, generating an electrochemical reagent comprises applying a constant voltage to at least one electrode, wherein the constant voltage has an absolute value of approximately 0.1 to 10 volts. Preferably, the voltage is an absolute value of approximately 1 to 5 volts. Preferably, the electrochemically-generated reagent is a base. Alternatively, the electrochemically-generated reagent is an acid. Preferably, the dehybridization solution comprises a buffer having a concentration of approximately 1 to 1000 millimolar and a pH of approximately 5 to 9. The buffer is selected from the group consisting of di-sodium phosphate, mono-sodium phosphate, citrate, carbonate, bicarbonate, borate, acetate, MES, Bis-Tris, ADA, ACES, PIPES, MOPSO, Bis-Tris Propane, BES, MOPS, HEPES, TES, DIPSO, TAPSO, TRIZMA, HEPPSO, POPSO, EPPS, TEA, Tricine, Bicine, TAPS, AMPSO, CHES, CAPSO, AMP, CAPS and combinations thereof. Preferably, the probe oligonucleotide is DNA. Alternatively, the probe oligonucleotide is RNA. Preferably, the target oligonucleotide is DNA. Alternatively, the target oligonucleotide is RNA.

The present invention further provides a method for selective dehybridization by an electrochemically-generated reagent on an electrode microarray for selective recovery of one or more target oligonucleotides. The electrode microarray has an electrode surface having at least a first electrode and a second electrode. The first electrode is proximate to a first porous reaction layer. The second electrode is proximate to a second porous reaction layer. A first probe oligonucleotide is bound to the first porous reaction layer. A second probe oligonucleotide is bound to the second porous reaction layer. A first target oligonucleotide is hybridized to the first probe oligonucleotide. A second target oligonucleotide is hybridized to the second probe oligonucleotide. The first target oligonucleotide and the first probe oligonucleotide form a first oligonucleotide duplex. The second target oligonucleotide and the second probe oligonucleotide form a second oligonucleotide duplex. An electrochemically-generated reagent, generated in a dehybridizing solution contacting the first porous reaction layer, the second porous reaction layer, and the electrode surface, dehybridizes the first oligonucleotide duplex. The electrochemically-generated reagent is generated by an activation means applied to the first electrode. The first target nucleotide is dehybridized and goes into the dehybridizing solution. The first probe oligonucleotide substantially remains attached to the first porous reaction layer. The second oligonucleotide duplex is not dehybridized.

Preferably, the first porous reaction layer is adsorbed to the first electrode and the second porous reaction layer is adsorbed to the second electrode. Alternatively, the first porous reaction layer is adsorbed or attached to an opposing surface to the electrode surface and the second porous reaction layer is attached to an opposing surface to the electrode surface. Preferably, the first probe oligonucleotide and the second probe oligonucleotide are synthesized in situ by an electrochemical synthesis means. Preferably, the electrochemical synthesis means comprises a phosphoramidite synthesis means and an electrochemical deblocking means. Alternatively, the first probe oligonucleotide and the second probe oligonucleotide are presynthesized and attached to the reaction layer.

Preferably, the activation means comprises applying a constant current to the first electrode having an absolute value of approximately 0.1 to 20 microampere per electrode. Alternatively, the activation means comprises applying a constant voltage to the at least one electrode having an absolute value of approximately 0.1 to 10 volts. Preferably, the voltage is an absolute value of approximately 1 to 5 volts. Preferably, the electrochemically-generated reagent is a base. Alternatively, the electrochemically-generated reagent is an acid. Preferably, the dehybridization solution comprises a buffer having a concentration of approximately 1 to 1000 millimolar and a pH of approximately 5 to 9. The buffer is selected from the group consisting of di-sodium phosphate, mono-sodium phosphate, citrate, carbonate, bicarbonate, borate, acetate, MES, Bis-Tris, ADA, ACES, PIPES, MOPSO, Bis-Tris Propane, BES, MOPS, HEPES, TES, DIPSO, TAPSO, TRIZMA, HEPPSO, POPSO, EPPS, TEA, Tricine, Bicine, TAPS, AMPSO, CHES, CAPSO, AMP, CAPS and combinations thereof. Preferably, the first probe oligonucleotide and the second probe oligonucleotide are DNA. Alternatively, the first probe oligonucleotide and the second probe oligonucleotide are RNA. Preferably, the first target oligonucleotide and the second target oligonucleotide are DNA. Alternatively, the first target oligonucleotide and the second target oligonucleotide are RNA.

The present invention further provides a method for selective dehybridization by an electrochemically-generated (ECG) reagent on an electrode microarray using a chemical dehybridizing solution while protecting a target oligonucleotide by an ECG reagent. An electrode microarray is provided having an electrode surface having at least a first electrode and a second electrode. The first electrode is proximate to a first porous reaction layer having a plurality of first oligonucleotide duplexes, and the second electrode is proximate to a second porous reaction layer having a plurality of second oligonucleotide duplexes.

Each of the plurality of first oligonucleotide duplexes comprise a first probe oligonucleotide and a first target oligonucleotide, and each of the plurality of second oligonucleotide duplexes comprise a second probe oligonucleotide and a second target oligonucleotide. At least one of the plurality of first duplexes is dehybridized by a chemical dehybridizing solution contacting the first porous reaction layer, the second porous reaction layer, and the electrode surface while electrochemically-generated reagent prevents dehybridization of the plurality of second oligonucleotide duplexes.

The protecting electrochemically-generated reagent is generated by an activation means applied to the second electrode. The unprotected first target oligonucleotide is dehybridized and goes into the dehybridization solution. The first probe oligonucleotide is protected by an ECD reagent and substantially remains attached to the first porous reaction layer. The plurality of second oligonucleotide duplexes remains hybridized and attached to the second porous reaction layer.

Preferably, the first porous reaction layer is attached to the first electrode, and the second porous reaction layer is attached to the second electrode. Alternatively, the first porous reaction layer is attached to an opposing surface to the electrode surface, and the second porous reaction layer is attached to an opposing surface to the electrode surface. Preferably, the first probe oligonucleotide and the second probe oligonucleotide are synthesized in situ by an electrochemical synthesis process. Preferably, the electrochemical synthesis process comprises a phosphoramidite synthesis process and an electrochemical deblocking process. Alternatively, the first probe oligonucleotide and the second probe oligonucleotide are presynthesized and attached to the porous reaction layer.

Preferably, the activation process comprises applying a constant current to the first electrode having an absolute value of approximately 0.1 to 20 microampere per electrode. Alternatively, the activation process comprises applying a constant voltage to the at least one electrode having an absolute value of approximately 0.1 to 10 volts. Preferably, the voltage is an absolute value of approximately 1 to 5 volts. Preferably, the electrochemically-generated reagent is a base. Alternatively, the electrochemically-generated reagent is an acid.

Preferably, the chemical dehybridization solution comprises (a) an aqueous solution of buffer from approximately 1 to 1000 millimolar; (b) an organic that alters pH of dehybridization in a concentration from approximately 0 to 100% of the saturation value of the organic; and (c) a pH modifying substance in an amount sufficient to adjust the pH to a value of approximately below 5.5 or above approximately 10.0. The buffer is selected from the group consisting of di-sodium phosphate, mono-sodium phosphate, citrate, carbonate, bicarbonate, borate, acetate, MES, Bis-Tris, ADA, ACES, PIPES, MOPSO, Bis-Tris Propane, BES, MOPS, HEPES, TES, DIPSO, TAPSO, TRIZMA, HEPPSO, POPSO, EPPS, TEA, Tricine, Bicine, TAPS, AMPSO, CHES, CAPSO, AMP, CAPS and combinations thereof. Preferably, the buffer is a phosphate buffer. Any buffer capable of controlling pH is suitable. Preferably, the buffer is an organic buffer and is selected from the group consisting of hydroquinone, catechol, p-aminophenol, o-pnenylenediamine, p-pnenylenediamine, and combinations thereof. Preferably, the organic buffer is hydroquinone. Any organic buffer that affects the pH of hybridization is suitable.

Preferably, the first probe oligonucleotide and the second probe oligonucleotide are DNA. Alternatively, the first probe oligonucleotide and the second probe oligonucleotide are RNA. Preferably, the first target oligonucleotide and the second target oligonucleotide are DNA. Alternatively, the first target oligonucleotide and the second target oligonucleotide are RNA.

The present invention further provides a method for selective dehybridization by electrochemically-generated (ECG) reagent on an electrode microarray using a chemical dehybridizing solution while protecting a target oligonucleotide by ECG reagent. An electrode-microarray is provided having an electrode surface having at least a first electrode and a second electrode. The first electrode is proximate to a first porous reaction layer and the second electrode is proximate to a second porous reaction layer. A first probe oligonucleotide is bound to the first porous reaction layer, and a second probe oligonucleotide is bound to the second porous reaction layer.

A first target oligonucleotide is hybridized to the first probe oligonucleotide, and a second target oligonucleotide is hybridized to the second probe oligonucleotide. The first target oligonucleotide and the first probe oligonucleotide form a first oligonucleotide duplex, and the second target oligonucleotide and the second probe oligonucleotide forms a second oligonucleotide duplex. The first oligonucleotide duplex is dehybridized by a chemical dehybridizing solution contacting the first porous reaction layer, the second reaction layer, and the electrode surface while electrochemically-generated reagent prevents dehybridization of the plurality of second oligonucleotide duplexes.

The electrochemically-generated reagent is generated by an activation process applied to the second electrode. The first target nucleotide is dehybridized and goes into the dehybridizing solution. The first probe oligonucleotide substantially remains attached to the first porous reaction layer. The second oligonucleotide duplex is not dehybridized.

Preferably, the first porous reaction layer is attached to the first electrode, and the second porous reaction layer is attached to the second electrode. Alternatively, the first porous reaction layer is attached to an opposing surface to the electrode surface, and the second porous reaction layer is attached to an opposing surface to the electrode surface. Preferably, the first probe oligonucleotide and the second probe oligonucleotide are synthesized in situ by an electrochemical synthesis process. Preferably, the electrochemical synthesis process comprises a phosphoramidite synthesis process and an electrochemical deblocking process. Alternatively, the first probe oligonucleotide and the second probe oligonucleotide are presynthesized and attached to the respective porous reaction layers.

Preferably, the activation process comprises applying a constant current to the first electrode having an absolute value of approximately 0.1 to 20 microampere per electrode. Alternatively, the activation process comprises applying a constant voltage to the at least one electrode having an absolute value of approximately 0.1 to 10 volts. Preferably, the voltage is an absolute value of approximately 1 to 5 volts. Preferably, the electrochemically-generated reagent is a base. Alternatively, the electrochemically-generated reagent is an acid.

Preferably, the chemical dehybridization solution a buffer solution selected from the group consisting of (a) an aqueous buffer from approximately 1 to 1000 millimolar; (b) an organic buffer that alters pH of dehybridization in a concentration from approximately 0 to 100% of the saturation value; and (c) a pH modifying substance in an amount sufficient to adjust the pH to a value of approximately below 5.5 or above approximately 10.0. The aquesou buffer is selected from the group consisting of di-sodium phosphate, mono-sodium phosphate, citrate, carbonate, bicarbonate, borate, acetate, MES, Bis-Tris, ADA, ACES, PIPES, MOPSO, Bis-Tris Propane, BES, MOPS, HEPES, TES, DIPSO, TAPSO, TRIZMA, HEPPSO, POPSO, EPPS, TEA, Tricine, Bicine, TAPS, AMPSO, CHES, CAPSO, AMP, CAPS and combinations thereof. Preferably, the aqueous buffer is a phosphate buffer. Any buffer capable of controlling pH is suitable. The organic buffer is selected from the group consisting of hydroquinone, catechol, p-aminophenol, o-pnenylenediamine, p-pnenylenediamine, and combinations thereof. Preferably, the organic buffer is hydroquinone. Any organic buffer that affects the pH of hybridization is suitable.

Preferably, the first probe oligonucleotide and the second probe oligonucleotide are DNA. Alternatively, the first probe oligonucleotide and the second probe oligonucleotide are RNA. Preferably, the first target oligonucleotide and the second target oligonucleotide are DNA. Alternatively, the first target oligonucleotide and the second target oligonucleotide are RNA.

The present invention further provides a method for selective dehybridization by electrochemically-generated reagent on an electrode microarray. The method comprises applying an activation process to a dehybridizing solution contacting an oligonucleotide duplex attached to a porous reaction layer proximate to an electrode on the electrode microarray. The dehybridizing solution comprises an aqueous solution of buffer from approximately 1 to 1000 millimolar at a pH of approximately 5 to 9.

Preferably, the dehybridization solution comprises a buffer having a concentration of approximately 1 to 1000 millimolar of buffer and a pH of approximately 5 to 9. The buffer is selected from the group consisting of di-sodium phosphate, mono-sodium phosphate, citrate, carbonate, bicarbonate, borate, acetate, MES, Bis-Tris, ADA, ACES, PIPES, MOPSO, Bis-Tris Propane, BES, MOPS, HEPES, TES, DIPSO, TAPSO, TRIZMA, HEPPSO, POPSO, EPPS, TEA, Tricine, Bicine, TAPS, AMPSO, CHES, CAPSO, AMP, CAPS and combinations thereof.

The present invention further provides a method is provided for selective dehybridization by electrochemically-generated reagent on an electrode microarray. The method comprises applying an activation means to a second electrode, wherein a chemical dehybridizing solution is contacting a first plurality of oligonucleotide duplexes attached to a first porous reaction layer proximate to a first electrode and a second plurality of oligonucleotide duplexes attached to a second porous reaction proximate to the second electrode. The chemical dehybridizing solution comprises a buffer selected from the group consisting of (a) an aqueous buffer from approximately 1 to 1000 millimolar; (b) an organic buffer in a concentration from approximately 0 to 100% of the saturation value of the organic buffer; and (c) a pH modifying substance in an amount sufficient to adjust the pH to a value of approximately below 5.5 or above approximately 10.0.

The aqueous buffer is selected from the group consisting of di-sodium phosphate, mono-sodium phosphate, citrate, carbonate, bicarbonate, borate, acetate, MES, Bis-Tris, ADA, ACES, PIPES, MOPSO, Bis-Tris Propane, BES, MOPS, HEPES, TES, DIPSO, TAPSO, TRIZMA, HEPPSO, POPSO, EPPS, TEA, Tricine, Bicine, TAPS, AMPSO, CHES, CAPSO, AMP, CAPS and combinations thereof. Preferably, the aqueous buffer is a phosphate buffer. Any buffer capable of controlling pH is suitable. The organic buffer is selected from the group consisting of hydroquinone, catechol, p-aminophenol, o-pnenylenediamine, p-pnenylenediamine, and combinations thereof. Preferably, the organic buffer is hydroquinone. Any organic buffer that affects the pH of hybridization is suitable.

### BRIEF DESCRIPTION OF THE DRAWINGS

FIG. 1 is a schematic of an electrode microarray having an oligonucleotide duplex undergoing ECG dehybridization. The microarray has at least one electrode having only one type of probe with a plurality of duplex on the electrode. The counter electrode is not shown but may be on the microarray or off the microarray.

FIG. 2 is a schematic of an electrode microarray having an oligonucleotide duplex undergoing ECG dehybridization. The microarray has at least one electrode and an opposing surface having a plurality of duplex undergoing the ECG dehybridization. The counter electrode is not shown but may be on the microarray or off the microarray.

FIG. 3 is a schematic of an electrode microarray having at least two electrodes having a plurality of different duplexes where at least one duplex undergoes ECG dehybridization. There are at least two different types of probes. The counter electrode is not shown but may be on the microarray or off the microarray.

FIG. 4 is a schematic of an electrode microarray having at least one electrode and an opposing surface having a plurality of duplex undergoing ECG dehybridization. There are at least two different types of probes. The counter electrode is not shown but may be on the microarray or proximate to the microarray.

FIG. 5 is a schematic of an electrode microarray showing a series of steps for attachment of a single probe followed by ECG dehybridization. The counter electrode is not shown but may be on the microarray or off the microarray.

FIG. 6 is a schematic of an electrode microarray showing a series of steps for the attachment of a single probe to an opposing surface followed by ECG dehybridization. The counter electrode is not shown but may be on the microarray or off the microarray.

FIG. 7 is a schematic of an electrode microarray showing a series of steps for the attachment of at least two different probes followed by ECG dehybridization of one target hybridized to one probe type. The counter electrode is not shown but may be on the microarray or off the microarray.

FIG. 8 is a schematic of an electrode microarray showing a series of steps for the attachment of at least two different probes to an opposing surface followed by ECG dehybridization of one target hybridized to one probe type. The counter electrode is not shown but may be on the microarray or off the microarray.

FIG. 9 is a schematic of an electrode microarray having at least two electrodes having two different probes forming duplexes. One duplex undergoes chemical dehybridization while ECG reagent protects the other duplex. The counter electrode is not shown but may be on the microarray or off the microarray.

FIG. 10 is a schematic of an electrode microarray having at least two electrodes and an opposing surface have two different probes forming duplexes. One duplex undergoes chemical dehybridization while ECG reagent protects the other duplex. The counter electrode is not shown but may be on the microarray or off the microarray.

FIG. 11 is a schematic of an electrode microarray having at least two electrodes. There is a series of steps that include attachment of at least two different probes followed by hybridization with targets. At least one duplex undergoes chemical dehybridization while ECG reagent protects the other duplex. The counter electrode is not shown but may be on the microarray or off the microarray.

FIG. 12 is a schematic of an electrode microarray having at least two electrodes and an opposing surface having reaction layers. There is a series of steps that include attachment of at least two different probes followed by hybridization with targets to the reaction layers. At least one duplex undergoes chemical dehybridization while ECG reagent protects the other duplex. The counter electrode is not shown but may be on the microarray or proximate to the microarray.

FIG. 13 is a black and white conversion of an original gray scale image of the top view of a portion of an electrode microarray after hybridization of the target oligonucleotide to the probe oligonucleotide. The target oligonucleotide has a fluorescent tag to allow viewing by a fluorescent imager.

FIG. 14 is a black and white representation of a fluorescent image of the top view of an electrode on a second microarray after exposing the microarray to a PBS solution that had been exposed to a first microarray without generation ECG reagent on the first microarray.

FIG. 15 is a black and white conversion of a gray scale image of an electrode microarray after electrochemical dehybridization by ECG base using different voltages.

FIG. 16 is a black and white conversion of a grays scale image of a single electrode on a microarray that was hybridized using a solution containing target oligonucleotide that was dehybridized from another microarray.

FIG. 17 is a black and white conversion of a gray scale image of an electrode microarray after electrochemical dehybridization by ECG base using different voltages and different exposure times.

FIG. 18 is a black and white conversion of a gray scale image of an electrode microarray after electrochemical dehybridization by ECG base using different voltages and different buffer solutions.

FIG. 19 is a black and white conversion of a gray scale image of an electrode microarray after electrochemical dehybridization by ECG base using different voltages and a different number of active electrodes.

FIG. 20 is a black and white conversion of a gray scale image of an electrode microarray after electrochemical dehybridization by ECG base using different voltages, a different number of active electrodes, and using a horizontal orientation for the microarray.

FIG. 21 is a black and white conversion of a gray scale image of an electrode microarray after electrochemical dehybridization by ECG base using different currents, a different number of active electrodes, and using a horizontal orientation for the microarray.

FIG. 22 is a black and white conversion of a gray scale image of an electrode microarray after rehybridization to the microarray having undergone dehybridization.

FIG. 23 is a plot of the concentration of hydroquinone as a percentage of saturation versus pH of a buffer solution showing the pH where dehybridization occurs.

FIG. 24 is black and white conversion of a gray scale image of portions of an electrode microarray before and after exposure to a dehybridization solution at different pH's.

FIG. 25 is a black and white conversion of a gray scale image of portions of an electrode microarray before and after exposure to a dehybridization solution while selected electrodes were protected by ECG base.

Definition List 1

| Term | Definition |
|---|---|
| Electrode Microarray | The term “electrode microarray” means a microarray of |
| | electrodes on a solid substrate. Each electrode is individually |
| | addressable and can be activated as an anode or as a cathode. |
| | The solid substrate has an electrode surface having the |
| | electrodes. Generally, the size of the electrodes is |
| | approximately 0.1-100 μm. The number of electrodes can be as |
| | few as one and up to tens of thousands and even hundreds of |
| | thousands. Generally, the size of an electrode microarray is not |
| | limited and neither is the number of electrodes except by |
| | practical utility. |
| Reaction layer | Generally, there is a reaction layer on the electrodes that allows |
| | electrochemical synthesis or attachment of pre-synthesized |
| | materials. DNA, RNA, and other molecules can be |
| | electrochemically synthesized in situ at the electrodes of an |
| | electrode microarray or attached to the microarray electrodes. |
| | Optionally, there is an opposing surface to the electrodes |
| | having the reaction layer where synthesis or attachment |
| | occurs. The opposing surface is held close to the electrode |
| | surface and is in electrical contact by a solution between the |
| | electrodes and the opposing surface. |
| Target oligonucleotide | Target oligonucleotide is a single strand of DNA or RNA. Target |
| | oligonucleotide is hybridized to a probe oligonucleotide on an |
| | electrode microarray. Target oligonucleotide is not attached to |
| | the microarray other than by hybridization to probe |
| | oligonucleotide. |
| Probe oligonucleotide | The term “probe oligonucleotide” means a single strand of DNA |
| | or RNA that usually is synthesized in situ on an electrode |
| | microarray. Probe oligonucleotide is substantially |
| | complementary to target oligonucleotide that hybridizes to the |
| | probe oligonucleotide. Probe oligonucleotide may be pre- |
| | synthesized and then attached to an electrode microarray. |
| | Probe DNA specifically refers to a DNA strand, and probe RNA |
| | specifically refers to a RNA strand. |
| Oligonucleotide | The term “oligonucleotide duplex” means a hybridized |
| duplex | structure of two oligonucleotides. The oligonucleotides are |
| | DNA, RNA, or a composite DNA/RNA. A hybridized structure |
| | comprising probe oligonucleotide and target oligonucleotide is |
| | an example of a oligonucleotide duplex. The target |
| | oligonucleotide of an oligonucleotide duplex is not attached to |
| | a microarray other than by hybridization to a probe |
| | oligonucleotide that is attached to an electrode microarray. |
| Dehybridizing Solution | The term “dehybridizing solution” means a solution that is in |
| | contact with an electrode microarray during electrochemical |
| | dehybridization. Preferably, the solution confines the |
| | electrochemically-generated reagent to the electrode that is |
| | making such reagent thus confining dehybridization to that |
| | electrode. The solution receives the target oligonucleotide that |
| | is dehybridized. The solution may be collected to recover |
| | dehybridized target oligonucleotide. |
| Chemical | The term “chemical dehybridizing solution” means a solution |
| dehybridizing solution | capable of causing dehybridization without application of a |
| | voltage or current to generate ECG reagent to cause |
| | dehybridization. An example is a buffer solution of citrate- |
| | phosphate saturated with hydroquinone and with hydrochloric |
| | acid to adjust pH to 5.5 or below. |
| Electrochemically- | The term “electrochemically-generated reagent” or “ECG |
| generated reagent | reagent” means a chemical species that is electrochemically- |
| | generated at an electrode by the application of a voltage or |
| | current. The electrochemical reaction is a non-spontaneous |
| | reaction. Generally, the reagent is an acid or a base although |
| | oxidants and reducing agents may be generated. |
| Activation means | The term “activation means” is where a voltage or current is |
| | applied to an electrode on an electrode microarray for a period |
| | of time. The voltage or current makes ECG reagent in sufficient |
| | amount to cause dehybridization of an oligonucleotide duplex |
| | on the electrode or in a sufficient amount to prevent |
| | dehybridization by a chemical dehybridization solution. |
| Buffer | The term “buffer” means any aqueous or organic solution |
| | having the ability to control pH or resist changes in pH. |
| | Examples of buffers include the following: di-sodium |
| | phosphate, mono-sodium phosphate, citrate, carbonate, |
| | bicarbonate, borate, acetate, 2-(N-morpholino)ethanesulfonic |
| | acid (MES), bis(2- |
| | hydroxyethyl)iminotris(hydroxymethyl)methane (Bis-Tris), N- |
| | (2-acetamido)-2-iminodiacetic acid (ADA), 2-[(2-amino-2- |
| | oxoethyl)amino]ethanesulfonic acid (ACES), piperaine-N,N′- |
| | bis(2-ethansulfonic acid (PIPES), 3-(N-morpholino)-2- |
| | hydroxypropanesulfonic acid (MOPSO), 1,3- |
| | bis[tris(hydroxymethyl)methylamino]propane (Bis-Tris |
| | Propane), N,N-bis(2-hydroxyethyl)-2-aminoethanesulfonic |
| | acid (BES), 3-(N-morpholino)propanesulfonic acid (MOPS), N- |
| | (2-hydroxyethyl)piperazine-N′-(2-ethanesulfonic acid) |
| | (HEPES), N-tris(hydroxymethyl)methyl-2-aminoethanesulfonic |
| | acid (TES), 3-[N,N-bis(2-hydroxyethyl)amino]-2- |
| | hydroxypropanesulfonic acid (DIPSO), 3-[N- |
| | tris(hydroxymethylamino]-2-hydroxypropanesulfonic acid |
| | (TAPSO), tris(hydroxymethyl)aminomethane (TRIZMA), N-(2- |
| | hydroxyethyl)piperazine-N′-(2-hydroxypropanesulfonic acid) |
| | (HEPPSO), piperazine-N,N′-bis(2-hydroxypropanesulfonic acid) |
| | (POPSO), N-(2-hydroxyethyl)piperazine-N′-(3-propanesulfonic |
| | acid) (EPPS), triethanolamine (TEA), N- |
| | tris(hydroxymethyl)methylglycine (Tricine), N,N-bis(2- |
| | hydroxyethyl)glycine (Bicine), N-tris(hydroxymethyl)methyl-3- |
| | aminopropanesulfonic acid (TAPS), 3-[(1,1-dimethyl-2- |
| | hydroxyethyl)amino]-2-hydroxypropanesulfonic acid (AMPSO), |
| | 2-(N-cyclohexylamino)ethanesulfonic acid (CHES), 3- |
| | (cyclohexylamino)-2-hydroxy-1-propanesulfonic acid (CAPSO), |
| | 2-amino-2-methyl-1-propanol (AMP), and 3- |
| | (cyclohexylamino)-1-propanesulfonic acid (CAPS). |

### DETAILED DESCRIPTION OF THE INVENTION

The present invention has application in DNA and RNA hybridization experiments such as in gene expression. For example, a probe DNA may be designed to hybridized to a specific gene or genetic marker. A dehybridized (denatured) DNA sample may be added to a microarray having probe DNA, including a probe for the gene or marker. The DNA sample may be allowed to hybridize to the microarray. The application of a voltage or a current to the electrode having the specific probe DNA of interest makes electrochemically-generated reagent that alters the pH at the electrode. The change in pH is sufficient to cause dehybridization at that specific electrode. For example, the pH may be increased sufficiently at a cathode to dehybridize DNA located at the cathode. The dehybridized DNA sequence is recovered and can be subsequently amplified by PCR and then analyzed further by chromatography or other methods.

Electrode Microarray Having Duplexes with One Probe Type

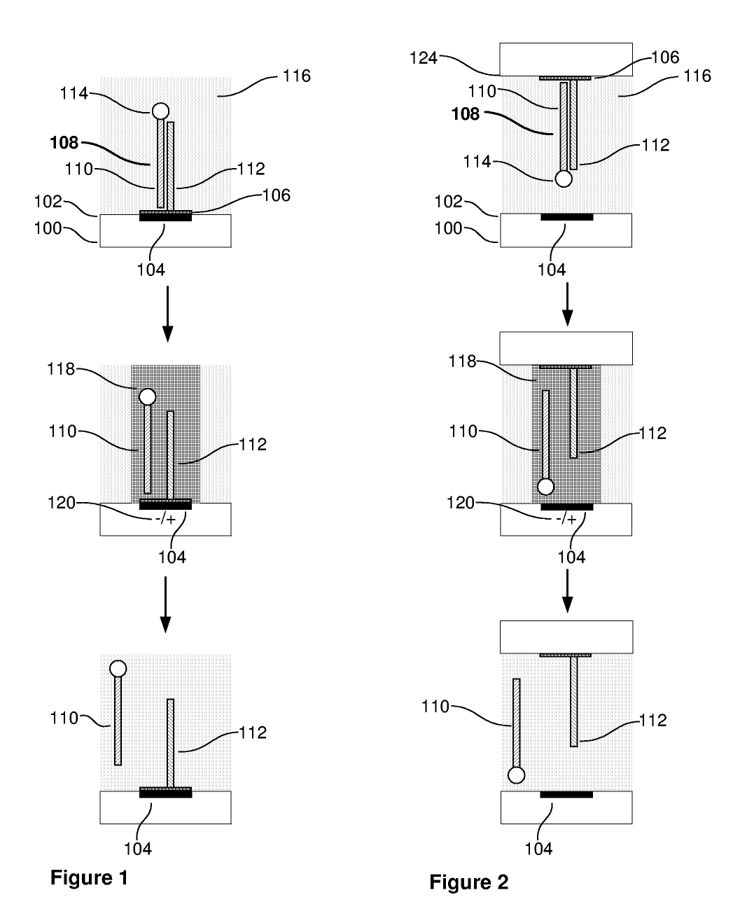

The present invention provides a method for selective dehybridization by electrochemically-generated reagent on an electrode microarray for recovery of a single type or sequence of hybridized target oligonucleotides. In particular, the method may be used to isolate one particular target oligonucleotide from a solution. The target is captured by a complementary probe oligonucleotide on a microarray. The solution is removed and then a new solution is added. Dehybridization is performed in the new solution to capture the target oligonucleotide. The target oligonucleotide can be recovered subsequently amplified by PCR and used in genomic studies. In FIG. 1, a schematic of an electrode microarray is provided according to the current embodiment of the present invention. Referring to FIG. 1, an electrode microarray 100 is provided having an electrode surface 102. The electrode surface 102 has at least one electrode 104 proximate to a reaction layer 106. Generally, electrode microarrays will have numerous electrodes where probes can capture targets.

The porous reaction layer has at least one oligonucleotide duplex 108, but generally, there will be a plurality of duplexes at each electrode 104. Each duplex 108 comprises a target oligonucleotide 110 and a probe oligonucleotide 112. The target oligonucleotide 110 is shown having an optional tag 114. Preferably, the optional tag is a fluorescent tag. A suitable tag is Cy5 DIRECT® as well as any other fluorescent tag. There is a dehybridizing solution 116 contacting the porous reaction layer 106 and the electrode surface 102.

An electrochemically-generated reagent 118 dehybridizes the duplex 108. The electrochemically-generated reagent 118 is generated by an activation means 120 applied to the electrode 104. The activation means is a constant voltage or current applied for a fixed period of time. The target oligonucleotide 110 goes into the dehybridizing solution 114. The probe nucleotide 112 substantially remains attached to the reaction layer 106. The Counter electrode may be off the microarray or on the microarray surface.

Preferably, the porous reaction layer 106 is attached directly to the electrode 104. Alternatively and referring to FIG. 2, the porous reaction layer 106 is attached to an opposing surface 124 to the electrode surface 102. The opposing surface 124 may be a planar surface that is parallel to the electrode surface 102 and sufficiently close to allow electrochemically-generated reagent 118 to reach the opposing surface 124. The opposing surface may be a point affixed to a substrate. The distance between the electrode surface 102 and the opposing surface 124 is approximately 0.1 to 1000 micrometers.

Preferably, the probe oligonucleotide 112 is synthesized in situ by an electrochemical synthesis means. Preferably, the electrochemical synthesis means comprises using standard a phosphoramidite synthesis means and an electrochemical deblocking means. The standard phosphoramidite synthesis means is the standard phosphoramidite synthesis for DNA and RNA. The electrochemical deblocking means comprises standard deblocking using ECG acid or base to remove protecting groups on the phosphoramidite monomers attached at each step of the synthesis. Alternatively, the probe oligonucleotide 112 is presynthesized and attached to the reaction layer 106.

Preferably, the activation means 120 comprises application of a constant current to the at least one electrode 104 of an absolute value of approximately 0.1 to 20 microampere per electrode. Alternatively, the activation means 120 comprises application of a constant voltage to the at least one electrode of an absolute value of approximately 0.1 to 10 volts. Preferably, the voltage is an absolute value of approximately 1 to 5 volts. Preferably, the number of electrodes 104 activated is at least one electrode. Preferably, the electrochemically-generated reagent 118 is a base. Alternatively, the electrochemically-generated reagent 118 is an acid.

Preferably, the dehybridization solution 116 comprises a buffer having a concentration of approximately 1 to 1000 millimolar of buffer and a pH of approximately 5 to 9. The buffer is selected from the group consisting of di-sodium phosphate, mono-sodium phosphate, citrate, carbonate, bicarbonate, borate, acetate, MES, Bis-Tris, ADA, ACES, PIPES, MOPSO, Bis-Tris Propane, BES, MOPS, HEPES, TES, DIPSO, TAPSO, TRIZMA, HEPPSO, POPSO, EPPS, TEA, Tricine, Bicine, TAPS, AMPSO, CHES, CAPSO, AMP, CAPS and combinations thereof. Any buffer of suitable concentration to confine the ECG reagent is appropriate for use in the present invention. More preferably, the dehybridization solution 116 comprises approximately 1 to 1000 millimolar di-sodium phosphate and mono-sodium phosphate to provide a pH of approximately 5 to 9. Preferably, the probe oligonucleotide 112 is DNA. Alternatively, the probe 112 oligonucleotide is RNA. Preferably, the target oligonucleotide 108 is DNA. Alternatively, the target oligonucleotide 108 is RNA.

Electrode Microarray Having Duplexes with at Least Two Probe Types

In another embodiment, the present invention provides a method for selective dehybridization by electrochemically-generated reagent on an electrode microarray for more than one target oligonucleotide. The method may be used to separate one particular target oligonucleotide from a mixture thereof by capturing the oligonucleotides on a microarray. After capture, the target oligonucleotides may be sequentially dehybridized into different solutions and subsequently amplified by PCR and used in genomic studies.

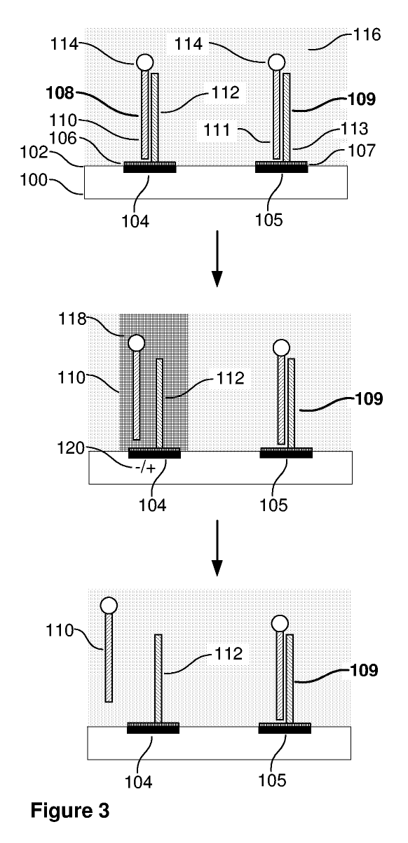

Referring to FIG. 3, a schematic of an electrode microarray 100 is provided having an electrode surface 102. The surface 102 has at least a first electrode 104 and a second electrode 105. The first electrode 104 is proximate to a first porous reaction layer 106 having a plurality of first oligonucleotide duplexes 108. The second electrode 105 is proximate to a second porous reaction layer 107 having a plurality of second oligonucleotide duplexes 109. Preferably, the porous reaction layers 106, 107 are the same matrix materials. Each of the plurality of first oligonucleotide duplexes 108 comprises a first probe oligonucleotide 112 and a first target oligonucleotide 110. Each of the plurality of second oligonucleotide duplexes 109 comprises a second probe oligonucleotide 113 and a second target oligonucleotide 111. The probe oligonucleotides have an optional tag 114. Preferably, the optional tag is a fluorescent tag. A suitable tag is Cy5 DIRECT® as well as any other fluorescent tag.

A dehybridizing solution 116 contacts the first reaction layer 106, the second porous reaction layer 107, and the electrode surface 102. Optionally, the porous reaction layer may be continuous and cover the entire electrode surface. At least one of the plurality of first duplexes 108 is dehybridized by an electrochemically-generated reagent 118. The electrochemically-generated reagent 118 is generated by an activation means 120 applied to the first electrode. The counter electrode is either located on the microarray or is located off the microarray but communicating with the dehybridization solution. The first target oligonucleotide 110 goes into the dehybridization solution 116. The first probe oligonucleotide 112 substantially remains attached to the first porous reaction layer 106. The plurality of second oligonucleotide duplexes 109 remains hybridized and attached to the second porous reaction layer 107.

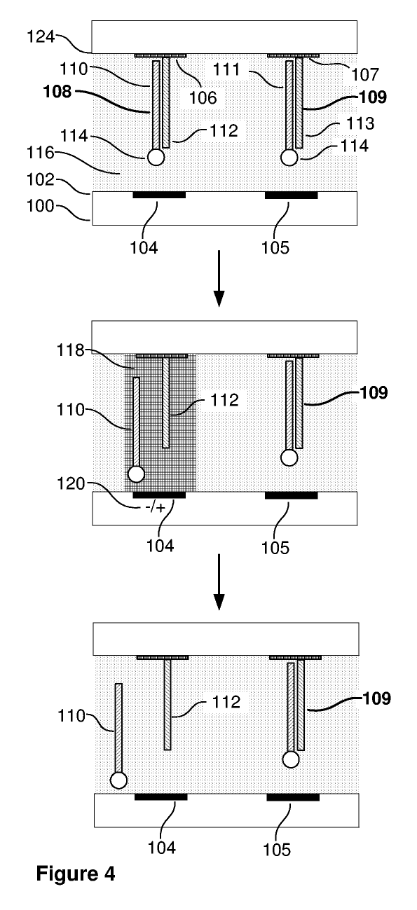

Preferably, the first porous reaction layer 106 is attached to the first electrode 104 and the second porous reaction layer 107 is attached to the second electrode 105. Alternatively and referring to FIG. 4, the first porous reaction layer 106 is attached to an opposing surface 124 to the electrode surface 102 and the second porous reaction layer 107 is attached to an opposing surface 124 to the electrode surface 102. The opposing surface 124 may be a surface that is approximately parallel to the electrode surface and sufficiently close to allow electrochemically-generated reagents 118 to diffuse to the opposing surface 124. Alternatively, the opposing surface may be a point affixed to a substrate. The distance between the electrode surface 102 and the opposing surface 124 is from about 0.1 to about 1000 micrometers.

Preferably, the first probe oligonucleotide 112 and the second probe oligonucleotide 113 are synthesized in situ by an electrochemical synthesis means. Preferably, the electrochemical synthesis means comprises a phosphoramidite synthesis means and an electrochemical deblocking means. The phosphoramidite synthesis means is based upon the original work of Caruthers phosphoramidite synthesis for DNA and RNA. Phosphoramidite reagents are available commercially having acid or base-cleavable protecting groups. The electrochemical deblocking means comprises deblocking using ECG acid or base to remove protecting groups on the phosphoramidite monomers attached at each stage of the synthesis. Alternatively, the first probe oligonucleotide 112 and the second probe oligonucleotide 113 are presynthesized and attached to their respective reaction layers 106, 107.

Preferably, the activation means 120 comprises application of a constant current to the first electrode 104 of an absolute value of approximately 0.1 to 20 microampere per electrode. Alternatively, the activation means 120 comprises application of a constant voltage to the first electrode 104 of an absolute value of approximately 0.1 to 10 volts. Preferably, the voltage is an absolute value of approximately 1 to 5 volts. Preferably, the electrochemically-generated reagent 118 is a base. Alternatively, the electrochemically-generated reagent 118 is an acid.

Preferably, the dehybridization solution 116 comprises a buffer (buffer) having a concentration of approximately 1 to 1000 millimolar of buffer and a pH of approximately 5 to 9. The buffer is selected from the group consisting of di-sodium phosphate, mono-sodium phosphate, citrate, carbonate, bicarbonate, borate, acetate, MES, Bis-Tris, ADA, ACES, PIPES, MOPSO, Bis-Tris Propane, BES, MOPS, HEPES, TES, DIPSO, TAPSO, TRIZMA, HEPPSO, POPSO, EPPS, TEA, Tricine, Bicine, TAPS, AMPSO, CHES, CAPSO, AMP, CAPS and combinations thereof. More preferably, the dehybridization solution 116 comprises approximately 1 to 1000 millimolar di-sodium phosphate and mono-sodium phosphate to provide a pH of approximately 5 to 9.

Preferably, the first probe oligonucleotide 112 and the second probe oligonucleotide 113 are DNA. Alternatively, the first probe oligonucleotide 112 and the second probe oligonucleotide 113 are RNA. Preferably, the first target oligonucleotide 110 and the second target oligonucleotide 111 are DNA. Alternatively, the first target oligonucleotide 110 and the second target oligonucleotide 111 are RNA. Each target oligonucleotide has an optional tag 114. Preferably, the optional tag is a fluorescent tag. A suitable tag is, for example, Cy5 DIRECT®.

Electrode Microarray Having One Probe Type

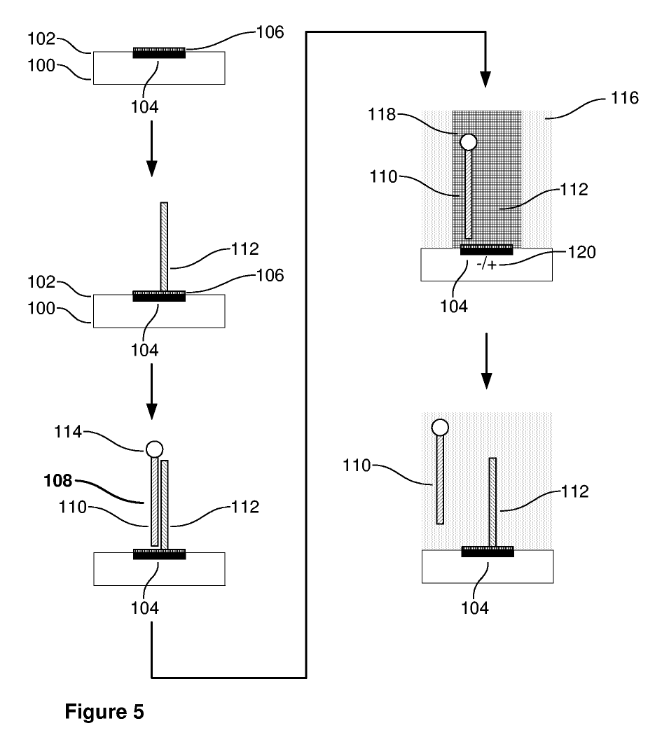

In an alternative embodiment, the present invention provides a method for selective dehybridization by electrochemically-generated reagent on an electrode microarray for recovery of a single type of target oligonucleotide. The method may be used to isolate one particular target oligonucleotide from a solution. The target is captured by a complementary probe oligonucleotide on a microarray. The solution is removed and then a new solution is added. Dehybridization is performed in the new solution to capture the target oligonucleotide. The target oligonucleotide can be recovered subsequently amplified by PCR and used in genomic studies. In FIG. 5, a schematic of an electrode microarray is provided according to this embodiment of the present invention. Referring to FIG. 5, an electrode microarray 100 is provided having an electrode surface 102 having at least one electrode 104 proximate to a reaction layer 106.

At least one probe oligonucleotide 112 is bound to the porous reaction layer 106. Generally, there are a plurality of nucleic acid duplex molecules at each electrode 104. A target oligonucleotide 110 is hybridized to the at least one probe oligonucleotide 112. The target oligonucleotide has an optional tag 114. Preferably, the optional tag is a fluorescent tag. A suitable tag is Cy5 DIRECT® as well as any other fluorescent tag. The target oligonucleotide 110 and the probe oligonucleotide 112 form an oligonucleotide duplex 108.

An electrochemically-generated reagent 118 dehybridizes the oligonucleotide duplex 108. The reagent 118 is generated in a dehybridizing solution 116 contacting the porous reaction layer 106 and the electrode surface 102. The porous reaction layer 106 may optionally cover the entire electrode surface 102. The electrochemically-generated reagent 118 is generated by an activation means 120 applied to the at least one electrode 104. The target nucleotide 110 goes into the dehybridizing solution 116. The probe oligonucleotide 112 substantially remains attached to the reaction layer 106. The dehybridizing solution 116, having the target nucleotide 110, is recovered.

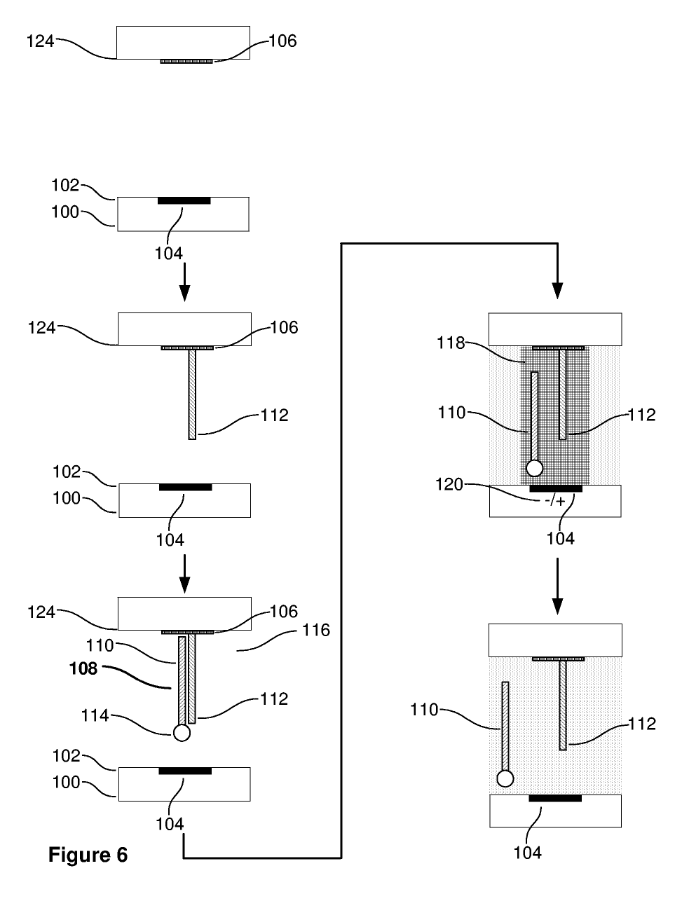

Preferably, the porous reaction layer 106 is attached directly to the electrode 104. Alternatively and referring to FIG. 6, the porous reaction layer 106 is attached to an opposing surface 124 to the electrode surface 102. The opposing surface 124 may be a planar surface that is approximately parallel to the electrode surface 102 and sufficiently close to allow electrochemically-generated reagent 118 to diffuse to the opposing surface 124. The opposing surface may be a point affixed to a substrate. The distance between the electrode surface 102 and the opposing surface 124 is approximately 0.1 to 1000 micrometers.

Preferably, the probe oligonucleotide 112 is synthesized in situ by an electrochemical synthesis reaction. Preferably, the electrochemical synthesis reaction comprises using a phosphoramidite synthesis chemistry (Caruthers) and an electrochemical deblocking solution. Phosphoramidite synthesis chemistry uses phosphoramidite nucleotides that are widely available commercially. The electrochemical deblocking solution comprises uses ECG acid or base to remove protecting groups on the phosphoramidite monomers. Alternatively, the probe oligonucleotide 112 is presynthesized and attached to the reaction layer 106.

Preferably, the activation means 120 comprises application of a constant current to the at least one electrode 104 of an absolute value of approximately 0.1 to 20 microampere per electrode. Alternatively, the activation means 120 comprises application of a constant voltage to the at least one electrode of an absolute value of approximately 0.1 to 10 volts. Preferably, the voltage is an absolute value of approximately 1 to 5 volts. Preferably, the number of electrodes 104 activated is at least one electrode. Preferably, the electrochemically-generated reagent 118 is a base. Alternatively, the electrochemically-generated reagent 118 is an acid.

Preferably, the dehybridization solution 116 comprises a buffer having a concentration of approximately 1 to 1000 millimolar of buffer and a pH of approximately 5 to 9. The buffer is selected from the group consisting of di-sodium phosphate, mono-sodium phosphate, citrate, carbonate, bicarbonate, borate, acetate, MES, Bis-Tris, ADA, ACES, PIPES, MOPSO, Bis-Tris Propane, BES, MOPS, HEPES, TES, DIPSO, TAPSO, TRIZMA, HEPPSO, POPSO, EPPS, TEA, Tricine, Bicine, TAPS, AMPSO, CHES, CAPSO, AMP, CAPS and combinations thereof. Any buffer of suitable concentration to confine the ECG reagent is appropriate for use in the present invention. More preferably, the dehybridization solution 116 comprises approximately 1 to 1000 millimolar di-sodium phosphate and mono-sodium phosphate to provide a pH of approximately 5 to 9. Preferably, the probe oligonucleotide 112 is DNA. Alternatively, the probe 112 oligonucleotide is RNA. Preferably, the target oligonucleotide 108 is DNA. Alternatively, the target oligonucleotide 108 is RNA.

Electrode Microarray Having at Least Two Probe Types

In an alternative embodiment, a method is provided for selective dehybridization by electrochemically-generated reagent on an electrode microarray for selective recovery of one or more target oligonucleotides. The method may be used to separate one particular target oligonucleotide from a mixture thereof by capturing the oligonucleotides on a microarray. After capture, the target oligonucleotides may be sequentially dehybridized into different solutions and subsequently amplified by PCR and used in genomic studies.

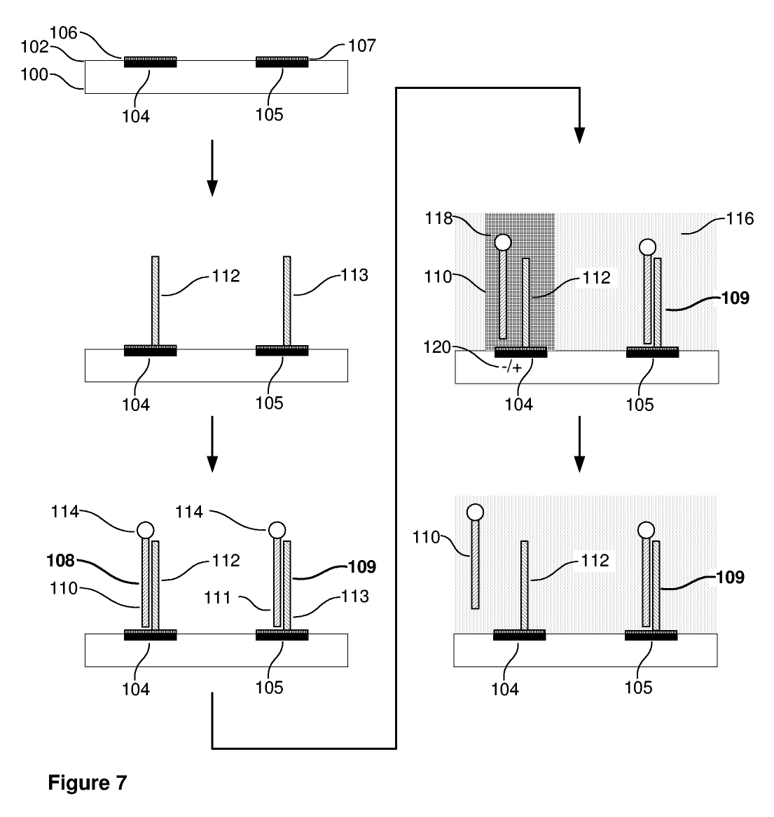

Referring to FIG. 7, a schematic of an electrode microarray 100 is provided having an electrode surface 102. The surface 102 has at least a first electrode 104 and a second electrode 105. The first electrode 104 is proximate to a first porous reaction layer 106. The second electrode 105 is proximate to a second porous reaction layer 107. A first probe oligonucleotide 112 is bound to the first porous reaction layer 106. A second probe oligonucleotide 113 is bound to the second porous reaction layer 107. Preferably, the porous reaction layer 106, 107 is the same matrix material. Optionally, the porous reaction layer may be continuous and cover the entire microarray surface. A first target oligonucleotide 110 is hybridized to the first probe oligonucleotide 112. A second target oligonucleotide 111 is hybridized to the second probe oligonucleotide 113. The first target oligonucleotide 110 and the first probe oligonucleotide 112 form a first oligonucleotide duplex 108. The second target oligonucleotide 111 and the second probe oligonucleotide 113 form a second oligonucleotide duplex 109. The probe oligonucleotides have an optional tag 114. Preferably, the optional tag is a fluorescent tag. A suitable fluorescent tag is, for example, Cy5 DIRECT®.

An electrochemically-generated reagent 118 is generated in a dehybridizing solution 116. The ECG reagent 118 contacts the only first porous reaction layer 106, but not the second porous reaction layer 107. The electrode surface 102 dehybridizes the first oligonucleotide duplex 108. The electrochemically-generated reagent 118 is generated by an activation means (i.e., voltage or current) applied to the first electrode 104. The counter electrode is located either on the microarray or is located off the microarray but communicating with the dehybridizing solution. The first target nucleotide 110 goes into the dehybridizing solution 116. The first probe oligonucleotide 112 substantially remains attached to the first reaction layer 106. The second oligonucleotide duplex 109 is not dehybridized.

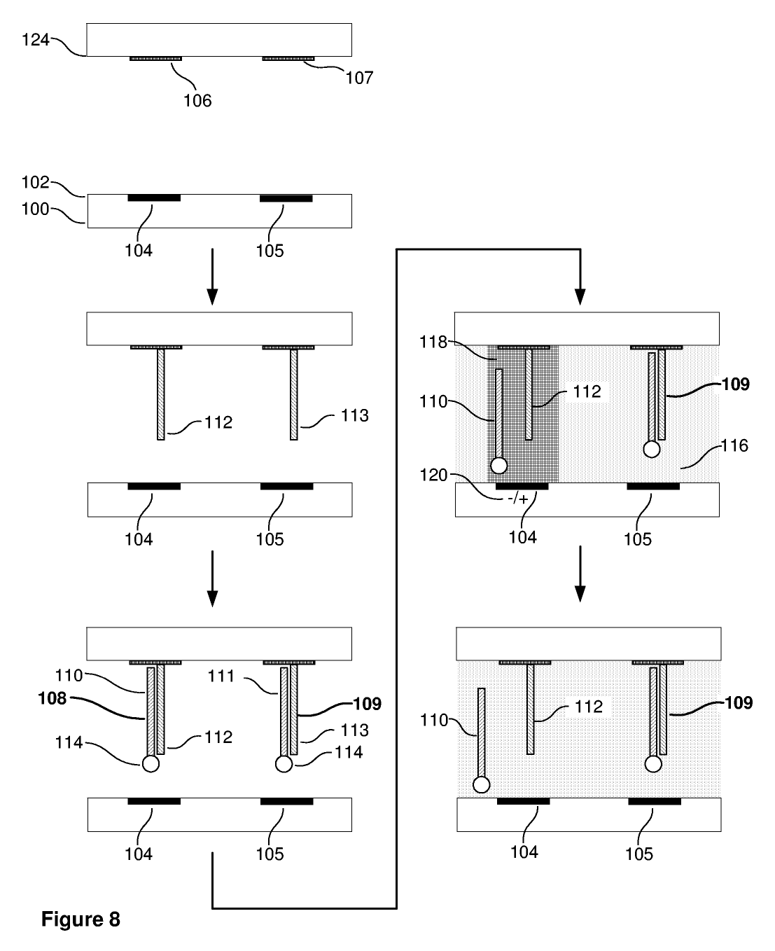

Preferably, the first porous reaction layer 106 is attached to the first electrode 104 and the second porous reaction layer 107 is attached to the second electrode 105. Alternatively and referring to FIG. 8, the first porous reaction layer 106 is attached to an opposing surface 124 to the electrode surface 102 and the second porous reaction layer 107 is attached to an opposing surface 124 to the electrode surface 102. The opposing surface 124 may be a surface that is approximately parallel to the electrode surface and sufficiently close to allow electrochemically-generated reagent 118 to diffuse the opposing surface 124. Alternatively, the opposing surface may be a point affixed to a substrate. The distance between the electrode surface 102 and the opposing surface 124 is approximately 0.1 to 1000 micrometers.

Preferably, the first probe oligonucleotide 112 and the second probe oligonucleotide 113 are synthesized in situ by an electrochemical synthesis means.

Preferably, the dehybridization solution 116 comprises a buffer having a concentration of approximately 1 to 1000 millimolar of buffer and a pH of approximately 5 to 9. The buffer is selected from the group consisting of di-sodium phosphate, mono-sodium phosphate, citrate, carbonate, bicarbonate, borate, acetate, MES, Bis-Tris, ADA, ACES, PIPES, MOPSO, Bis-Tris Propane, BES, MOPS, HEPES, TES, DIPSO, TAPSO, TRIZMA, HEPPSO, POPSO, EPPS, TEA, Tricine, Bicine, TAPS, AMPSO, CHES, CAPSO, AMP, CAPS and combinations thereof. More preferably, the dehybridization solution 116 comprises approximately 1 to 1000 millimolar di-sodium phosphate and mono-sodium phosphate to provide a pH of approximately 5 to 9.

Preferably, the first probe oligonucleotide 112 and the second probe oligonucleotide 113 are DNA. Alternatively, the first probe oligonucleotide 112 and the second probe oligonucleotide 113 are RNA. Preferably, the first target oligonucleotide 110 and the second target oligonucleotide 111 are DNA. Alternatively, the first target oligonucleotide 110 and the second target oligonucleotide 111 are RNA. Each target oligonucleotide has an optional tag 114. Preferably, the optional tag is a fluorescent tag. A suitable tag is, for example, Cy5 DIRECT®.

Chemical Dehybridization on an Electrode Microarray Having at Least Two Duplexes

In an alternative embodiment, a method is provided for selective dehybridization by electrochemically-generated reagent on an electrode microarray using a chemical dehybridizing solution while protecting a target oligonucleotide by ECG reagent. The method may be used to dehybridize an array while selectively protecting some electrodes from dehybridization. The dehybridized target oligonucleotides may be recovered and amplified by PCR for genomic studies.

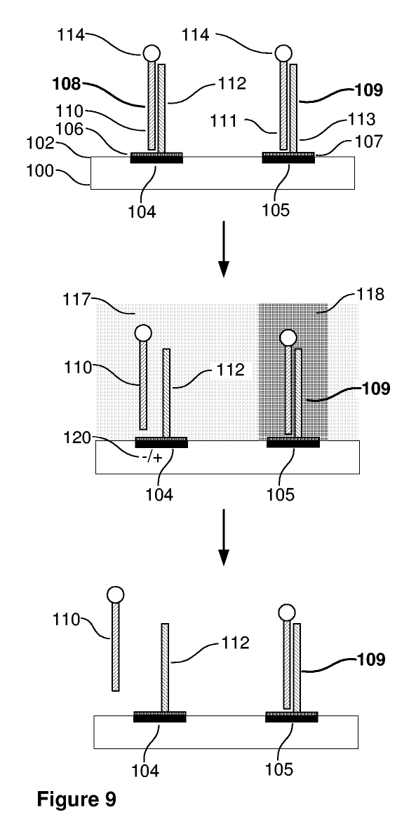

Referring to FIG. 9, an electrode microarray 100 is provided having an electrode surface 102 having at least a first electrode 104 and a second electrode 105. The first electrode 104 is proximate to a first porous reaction layer 106 having a plurality of first oligonucleotide duplexes 108, and the second electrode 105 is proximate to a second porous reaction layer 107 having a plurality of second oligonucleotide duplexes 109. Each of the plurality of first oligonucleotide duplexes 108 comprise a first probe oligonucleotide 112 and a first target oligonucleotide 110, and each of the plurality of second oligonucleotide duplexes 109 comprise a second probe oligonucleotide 113 and a second target oligonucleotide 111. The probe oligonucleotides have an optional tag 114. Preferably, the optional tag is a fluorescent tag. A suitable tag is, for example, Cy5 DIRECT®.

At least one of the plurality of first duplexes 108 is dehybridized by a chemical dehybridizing solution 117 contacting the first porous reaction layer 106, the second porous reaction layer 107, and the electrode surface 102 while electrochemically-generated reagent 118 prevents dehybridization of the plurality of second oligonucleotide duplexes 109. The electrochemically-generated reagent 118 is generated by an activation means 120 applied to the second electrode 105. The first target oligonucleotide 110 goes into the dehybridization solution 117. The first probe oligonucleotide 109 substantially remains attached to the first porous reaction layer 106. The plurality of second oligonucleotide duplexes 109 remains hybridized and attached to the second porous reaction layer 107. Preferably, the reaction layers 106, 107 are the same material. Optionally, the reaction layer may be continuous and cover the entire microarray surface 102.

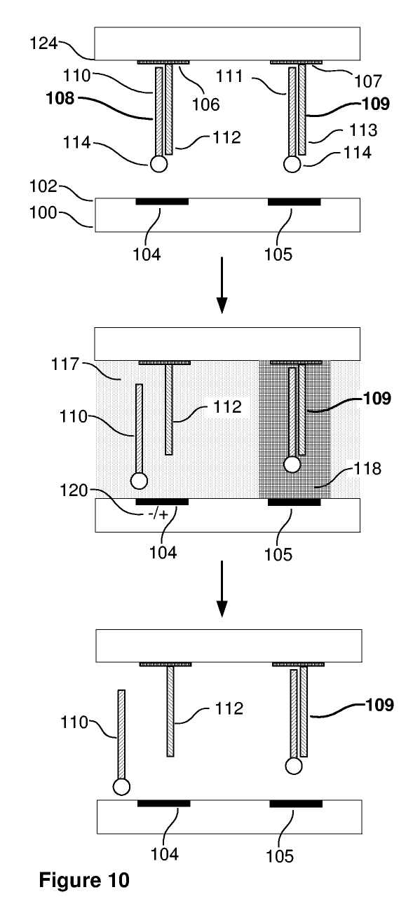

Preferably, the first reaction layer 106 is attached to the first electrode 104 and the second reaction layer 107 is attached to the second electrode 105. Alternatively and referring to FIG. 10, the first porous reaction layer 106 is attached to an opposing surface 124 to the electrode surface 102 and the second porous reaction layer 107 is attached to an opposing surface 124 to the electrode surface 102. The opposing surface 124 may be a planar surface that is parallel to the electrode surface and sufficiently close to allow electrochemically-generated reagent 118 to reach the opposing surface 124. Alternatively, the opposing surface may be a point affixed to a substrate. The distance between the electrode surface 102 and the opposing surface 124 is approximately 0.1 to 1000 micrometers.

Preferably, the first probe oligonucleotide 112 and the second probe oligonucleotide 113 are synthesized in situ by an electrochemical synthesis means. Preferably, the electrochemical synthesis means comprises a phosphoramidite synthesis means and an electrochemical deblocking means. The standard phosphoramidite synthesis means is the standard phosphoramidite synthesis for DNA and RNA. The electrochemical deblocking means comprises standard deblocking using ECG acid or base to remove protecting groups on the phosphoramidite monomers attached at each stage of the synthesis. Alternatively, the first probe oligonucleotide 112 and the second probe oligonucleotide 113 are presynthesized and attached to their respective porous reaction layers 106, 107.

Preferably, the activation means 120 comprises application of a constant current to the first electrode 104 of an absolute value of approximately 0.1 to 20 microampere per electrode. Alternatively, the activation means 120 comprises application of a constant voltage to the first electrode 104 of an absolute value of approximately 0.1 to 10 volts. Preferably, the voltage is an absolute value of approximately 1 to 5 volts. Preferably, the electrochemically-generated reagent 118 is a base. Alternatively, the electrochemically-generated reagent 118 is an acid.

Preferably, the chemical dehybridization solution 117 comprises (a) an aqueous solution of sodium phosphate from approximately 1 to 1000 millimolar; (b) hydroquinone in a concentration from approximately 0 to 100% of the saturation value of hydroquinone; and (c) a pH modifying substance in an amount sufficient to adjust the pH to a value of approximately below 5.5 or above approximately 10.0. Materials that affect the pH of hybridization are suitable replacements for hydroquinone.

Preferably, the first probe oligonucleotide 112 and the second probe oligonucleotide 113 are DNA. Alternatively, the first probe oligonucleotide 112 and the second probe oligonucleotide 113 are RNA. Preferably, the first target oligonucleotide 110 and the second target oligonucleotide 111 are DNA. Alternatively, the first target oligonucleotide 110 and the second target oligonucleotide 111 are RNA. Each target oligonucleotide has an optional tag 114. Preferably, the optional tag is a fluorescent tag. A suitable tag is, for example, Cy5 DIRECT®.

Chemical Dehybridization on an Electrode Microarray Having at Least Two Probes

In an alternative embodiment, a method is provided for selective dehybridization by electrochemically-generated reagent on an electrode microarray using a chemical dehybridizing solution while protecting a target oligonucleotide by ECG reagent. The method may be used to dehybridize an array while selectively protecting some electrodes from dehybridization. The dehybridized target oligonucleotides may be recovered and amplified by PCR for genomic studies.

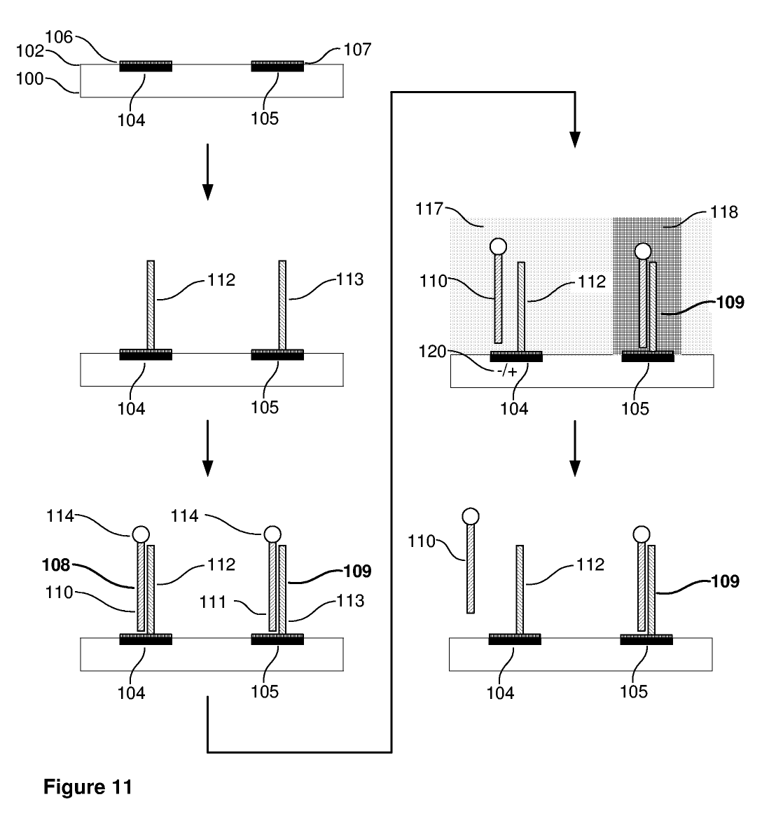

Referring to FIG. 11, an electrode-microarray 100 is provided having an electrode surface 102 having at least a first electrode 104 and a second electrode 105. The first electrode 104 is proximate to a first porous reaction layer 106 and the second electrode 105 is proximate to a second porous reaction layer 107. A first probe oligonucleotide 112 is bound to the first porous reaction layer 106, and a second probe oligonucleotide 113 is bound to the second porous reaction layer 107. Preferably, the porous reaction layers 106, 107 are the same matrix material. Optionally, the porous reaction layer may cover the entire electrode surface 102.

A first target oligonucleotide 110 is hybridized to the first probe oligonucleotide 112, and a second target oligonucleotide 111 is hybridized to the second probe oligonucleotide 113. Each target oligonucleotide has an optional tag 114. Preferably, the optional tag is a fluorescent tag. A suitable tag is, for example, Cy5 DIRECT®. The first target oligonucleotide 110 and the first probe oligonucleotide form 112 a first oligonucleotide duplex 108, and the second target oligonucleotide 111 and the second probe oligonucleotide 113 form a second oligonucleotide duplex 109. The first oligonucleotide duplex 108 is dehybridized by a chemical dehybridizing solution 117 contacting the first reaction layer 106, the second reaction layer 107, and the electrode surface 102 while electrochemically-generated reagent 118 prevents dehybridization of the plurality of second oligonucleotide duplexes 109.

The electrochemically-generated reagent 118 is generated by an activation means 120 applied to the second electrode. The counter electrode may be located on the microarray or located off the microarray but communicating with the dehybridizing solution. The first target nucleotide 110 is recovered. The first probe oligonucleotide 112 substantially remains attached to the first reaction layer 106. The second oligonucleotide duplex 109 is not dehybridized.

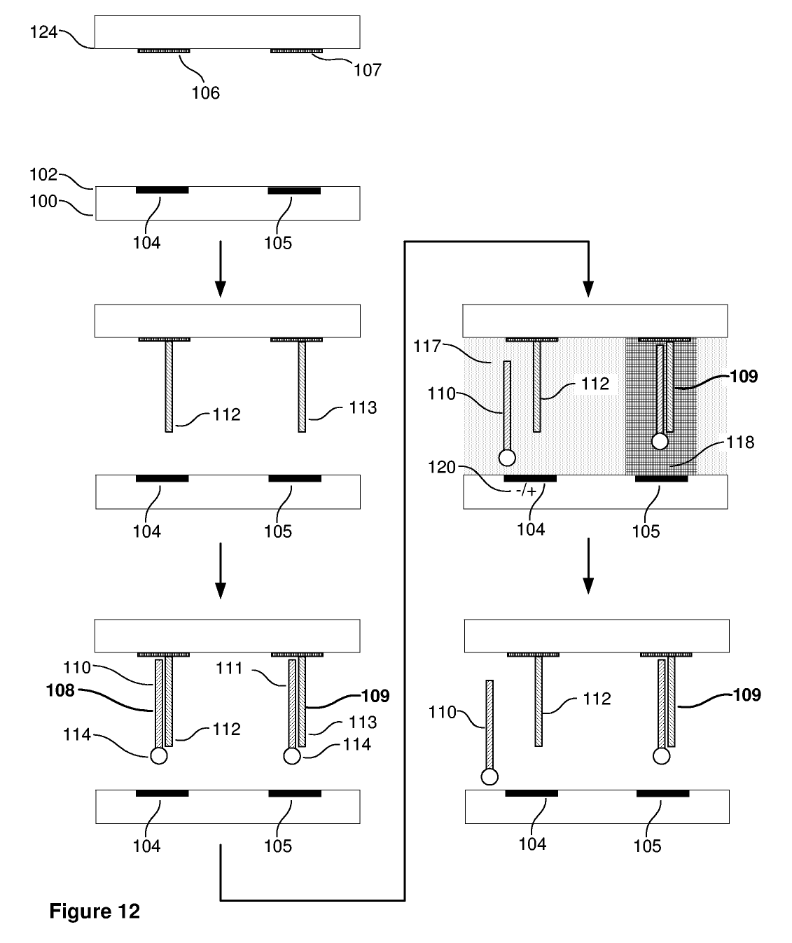

Preferably, the first porous reaction layer 106 is attached to the first electrode 104 and the second porous reaction layer 107 is attached to the second electrode 105. Alternatively and referring to FIG. 12, the first porous reaction layer 106 is attached to an opposing surface 124 to the electrode surface 102 and the second porous reaction layer 107 is attached to an opposing surface 124 to the electrode surface 102. The opposing surface 124 may be a planar surface that is approximately parallel to the electrode surface and sufficiently close to allow electrochemically-generated reagent 118 to diffuse to the opposing surface 124. Alternatively, the opposing surface may be a point affixed to a substrate. The distance between the electrode surface 102 and the opposing surface 124 is approximately 0.1 to 1000 micrometers.

Preferably, the first probe oligonucleotide 112 and the second probe oligonucleotide 113 are synthesized in situ by an electrochemical synthesis means. Alternatively, the first probe oligonucleotide 112 and the second probe oligonucleotide 113 are presynthesized and attached to their respective reaction layers 106, 107.

Preferably, the activation means 120 comprises application of a constant current to the first electrode 104 of an absolute value of approximately 0.1 to 20 microampere per electrode. Alternatively, the activation means 120 comprises application of a constant voltage to the first electrode 104 of an absolute value of approximately 0.1 to 10 volts. Preferably, the voltage is an absolute value of approximately 1 to 5 volts. Preferably, the electrochemically-generated reagent 118 is a base. Alternatively, the electrochemically-generated reagent 118 is an acid.

Preferably, the chemical dehybridization solution 117 comprises (a) an aqueous solution of sodium phosphate from approximately 1 to 1000 millimolar; (b) hydroquinone in a concentration from approximately 0 to 100% of the saturation value of hydroquinone; and (c) a pH modifying substance in an amount sufficient to adjust the pH to a value of approximately below 5.5 or above approximately 10.0. Materials that affect the pH of hybridization are suitable replacements for hydroquinone.

Preferably, the first probe oligonucleotide 112 and the second probe oligonucleotide 113 are DNA. Alternatively, the first probe oligonucleotide 112 and the second probe oligonucleotide 113 are RNA. Preferably, the first target oligonucleotide 110 and the second target oligonucleotide 111 are DNA. Alternatively, the first target oligonucleotide 110 and the second target oligonucleotide 111 are RNA. Each target oligonucleotide has an optional tag 114. Preferably, the optional tag is a fluorescent tag. A suitable tag is, for example, Cy5 DIRECT®.

Methods Using Solutions

In an alternative embodiment, a method is provided for selective dehybridization by electrochemically-generated reagent on an electrode microarray. The method comprises applying an activation means to a dehybridizing solution contacting an oligonucleotide duplex attached to a porous reaction layer proximate to an electrode on the electrode microarray. The solutions comprises an aqueous solution of sodium phosphate from approximately 1 to 1000 millimolar and a pH modifying substance in an amount sufficient to adjust the pH to a value of approximately 5 to 9.

In an alternative embodiment, a method is provided for selective dehybridization by electrochemically-generated reagent on an electrode microarray. The method comprises applying an activation means to a chemical dehybridizing solution contacting a first plurality of oligonucleotide duplexes attached to a first porous reaction layer proximate to a first electrode and a second plurality of oligonucleotide duplexes attached to a second porous reaction lay proximate to a second electrode comprising: (a) an aqueous solution of sodium phosphate from approximately 1 to 1000 millimolar; (b) hydroquinone in a concentration from approximately 0 to 100% of the saturation value of hydroquinone; and (c) a pH modifying substance in an amount sufficient to adjust the pH to a value of approximately below 5.5 or above approximately 10.0.

The following examples are provided merely to explain, illustrate, and clarify the present invention and not to limit the scope or application of the present invention.

### EXAMPLE 1

This example provides a description of selective electrochemical dehybridization of a double stranded deoxyribonucleic acid duplex comprising target oligonucleotide and probe oligonucleotide on an electrode microarray. The electrochemical dehybridization resulted from electrochemically-generated (ECG) reagent at selected electrodes that were used as cathodes. The ECG reagent was base that increased pH sufficiently to cause dehybridization. The microarray had the target oligonucleotide hybridized to probe oligonucleotide, which was electrochemically synthesized in situ on the electrode microarray. Specifically, a probe oligonucleotide was a DNA unit. The probe oligonucleotide comprised forty-five nucleic acid units (45-mer) and was synthesized in situ on the electrode microarray. The electrode microarray was a CombiMatrix Corporation CUSTOMARRAY 12K™. Each electrode on the microarray had the same probe oligonucleotide synthesized thereon; the probe oligonucleotide had sequence 5′ cataacgatggtggcgatgttaacctcggcttatggcgtcaccgg 3′ [SEQ ID NO:1].

To synthesize the probe oligonucleotide, an electrode microarray was provided having a porous reaction layer on the electrodes. The electrodes were platinum coated. The porous reaction layer matrix was sucrose. The probe oligonucleotide was synthesized 3′ to 5′, where the 3′ end was attached to the electrode microarray. Standard phosphoramidite chemistry was used to synthesize the probe oligonucleotide coupled with electrochemical deblocking to remove the protecting group for the addition of each subsequent oligonucleotide unit (A, C, G, or T/U.) For each electrochemical-deblocking step, a constant current of 0.26 microampere per electrode was applied for 60 seconds. The deblocking solution comprised 1 M hydroquinone, 10 mM benzoquinone, 50 mM tetraethyl ammonium p-toluene sulfonate, 5 mM 2,6-lutidine, 20% methanol, and 80% acetonitrile.

A solution having target oligonucleotide was made comprising target oligonucleotide having sequence 5′ ccggtgacgccataagccgaggttaacatcgccaccatcgttatg 3′, which is the exact complement of the probe oligonucleotide [SEQ ID NO:1]. The concentration of the target oligonucleotide in solution was 30 nanomolar. The solution was 3×SSPE buffer with 0.1% TWEEN®. The 3×SSPE solution comprised 450 mM sodium chloride, 30 mM sodium phosphate, and 3 mM EDTA and was obtained from Ambion, Inc. The target oligonucleotide had a Cy5 DIRECT® fluorescent label attached to the 3′ end. The solution was contacted to the electrode microarray to hybridize the target oligonucleotide to the probe oligonucleotide. The hybridization conditions comprised 55 degrees centigrade for 30 minutes.

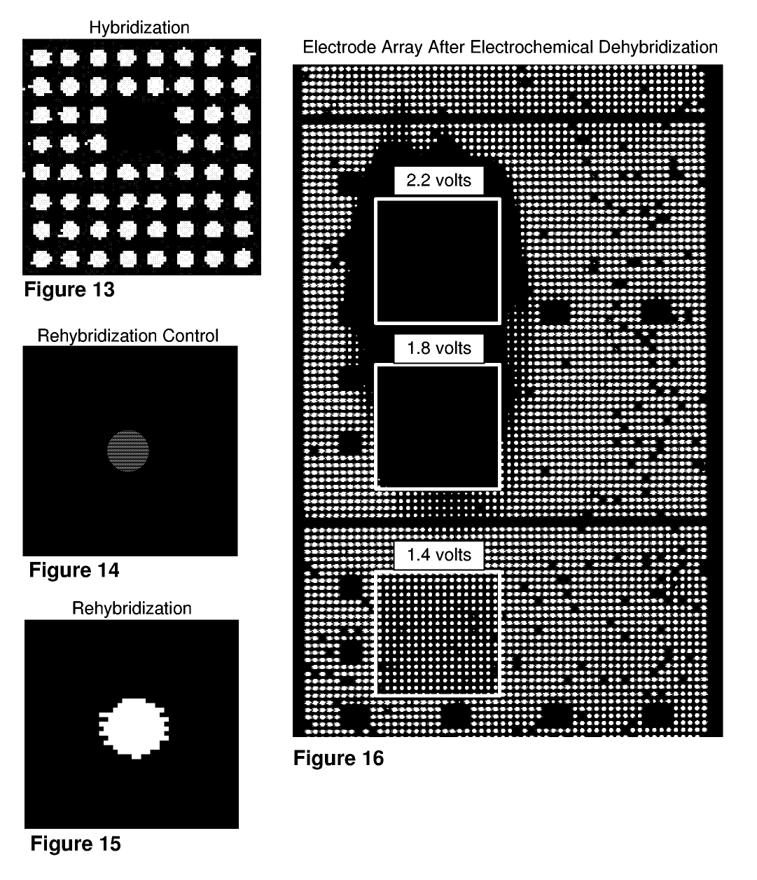

After hybridization, the microarray was viewed under a fluorescent imager to assess the degree of hybridization. The fluorescent imager used for this experiment was an Axon Instruments Genepix™ 4000B. FIG. 13 is a black and white conversion of an original gray scale image of the top view of a portion of an electrode microarray after hybridization of the target oligonucleotide to the probe oligonucleotide. The white spots represent the imaging of the Cy5 DIRECT® on the target oligonucleotide and correspond to the electrodes having the probe oligonucleotide. The black area in the middle of FIG. 13 represents four electrodes where the probe oligonucleotide was not synthesized and thus no target oligonucleotide hybridized to those electrodes. The ragged edge of the circular white spots is an artifact of the conversion from a gray scale image to a pure black and white image and also from saturation by use of excess target oligonucleotide.

Before dehybridization of the target oligonucleotide to the probe oligonucleotide, the electrode microarray was contacted to a solution of phosphate buffered saline (PBS). The purpose of such contact was to determine the amount, if any, of target oligonucleotide that will dehybridize from the probe oligonucleotide without the application of voltage or current to the electrodes as is required for electrochemical dehybridization. In essence, the contact to the PBS solution is a control for the electrochemical dehybridization. The PBS solution comprised 10 mM sodium phosphate, 150 mM sodium chloride. The PBS solution was recovered after being in contact with the microarray.

The recovered PBS solution was contacted to a second electrode microarray having probe oligonucleotide synthesized thereon. The probe oligonucleotide on the second electrode microarray was synthesized in situ and was complementary to the target oligonucleotide. If a significant amount of target oligonucleotide had come off from the first electrode microarray, then the second microarray would have fluorescence at those electrodes having probe oligonucleotide. The results of the experiment showed that an insignificant amount of target oligonucleotide came off the first microarray into the PBS solution. FIG. 14 is a black and white representation of a fluorescent image of the second microarray. The actual image of the electrodes showed a gray scale image that was nearly as dark as the surrounding non-electrode area. The computer screen displayed image of FIG. 14 shows a spottiness that was not present in the original gray scale image. The spottiness is a representation of an insignificant amount of Cy5 DIRECT® at the electrode and not an indication of spotty hybridization. The print image of FIG. 14 more accurately depicts the original gray scale image.

After determining that an insignificant amount of target oligonucleotide dehybridized when a microarray was exposes to PBS without an applied voltage or current, the first electrode array was re-contacted to fresh PBS solution of the same composition. Selected groups of electrodes were activated as cathodes. The activation comprised a pulse that lasted 10 seconds for each electrode group. The voltage potential for each group was set at 1.4, 1.8, or 2.2 volts. The number of electrodes in a group was 289 (17 by 17 square.) The counter electrode, the anode, was not on the microarray but was an off-array Pt electrode. During each pulse, the orientation of the electrode microarray was vertical.

As cathodes, the electrodes were expected to generate base, e.g., hydroxyl groups. The base was expected to cause dehybridization of the target oligonucleotide thus releasing the target oligonucleotide into the PBS solution. To indicate dehybridization, a loss of fluorescence was expected to occur at the activated electrodes because the target oligonucleotide had the Cy5 DIRECT® fluorescent tag. The target oligonucleotide was expected to dehybridize from activated electrodes while the probe oligonucleotide was expected to remain attached.

As expected, there was a loss of fluorescence at the activated electrodes as shown in FIG. 15, which is a black and white conversion of a gray scale image of the electrode microarray. The white spots represent the fluorescent tag on the electrodes. The white squares were added to the original figure in order to identify the electrodes undergoing dehybridization. FIG. 15 shows that at 1.8 and at 2.2 volts, there was essentially a complete loss of fluorescence on the activated electrodes. In the original gray scale image, some of the electrodes within the 1.8 and 2.2 volt squares had some fluorescence, which indicates incomplete dehybridization at those electrodes. Such electrodes were near the periphery of each square. FIG. 15 shows that at 1.4 volts, there was minimal dehybridization as indicated by minimal loss of fluorescence.

FIG. 15 shows that the electrode microarray had three by three and three by four electrode groupings where no probe oligonucleotide was synthesized. Thus, these electrode groupings remained dark as shown in the figure. Additionally, some random single electrodes shown in FIG. 15 are dark. The darkness is an artifact of the particular hybridization cap used on this electrode microarray. The grayscale image shows these electrodes as having some fluorescence.

One additional feature of FIG. 15 is that the base generated at the electrode groupings set at 1.8 and 2.2 volts caused dehybridization on nearby electrodes that were not activated. This dehybridization is a result of lack of containment of the ECG reagent (base) to the active electrodes. This lack of containment of the base shows that a different solution is required to better confine the ECG base to the active electrodes. Additionally the vertical orientation of the microarray resulted in more dehybridization in nearby inactive electrodes immediately above the activated electrodes. This effect of orientation shows that a horizontal orientation is more desirable for confinement of the electrochemically-generated base.

The PBS solution containing the target oligonucleotide after electrochemical dehybridization was recovered. This solution was contacted to a third microarray having probe oligonucleotide that was complementary to the target oligonucleotide. The probe oligonucleotide was electrochemically synthesized in situ on the electrode microarray. The target oligonucleotide in solution was hybridized to the probe oligonucleotide on the third microarray. The conditions for hybridization comprised 55 degrees Celsius for 30 minutes.

If the target oligonucleotide was dehybridized from the first electrode microarray as expected, then the target oligonucleotide should have been in the recovered solution. This recovered target oligonucleotide was expected to hybridize to the probe oligonucleotide of the third microarray. Such hybridization would have been shown by the existence of fluorescence on the electrodes having the probe oligonucleotide because the target oligonucleotide has the Cy5 DIRECT® tag.

As expected, the recovered PBS solution contained dehybridized target oligonucleotide as evidence by fluorescence at the electrodes on the third microarray having probe oligonucleotide. FIG. 16 is a black and white depiction of a typical gray scale image of an electrode having probe oligonucleotide. The white indicates hybridization of the recovered target oligonucleotide (DNA) from the recovered hybridization solution to the probe oligonucleotide on the third electrode microarray. Thus, the ECG base was effective at causing dehybridization on the first electrode microarray as evidenced by the fluorescent signal on the third electrode microarray.

The results of this experiment indicate that ECG reagent will dehybridize DNA duplex strands on an electrode microarray and allow recovery of the dehybridized single strand of DNA. However, in this experiment, confinement of the ECG reagent to the active electrodes was not sufficient to prevent dehybridization on adjacent non-active electrodes. Also, dehybridization was not complete on all activated electrodes. Parameters affecting confinement and dehybridization include, for example, buffer solution type and concentration, voltage or current strength, electrode on-times, total current on-times, and the number of electrodes turned on at one time.

### EXAMPLE 2

This example provides a description of constant voltage selective electrochemical dehybridization of a double stranded deoxyribonucleic acid duplex comprising target oligonucleotide and probe oligonucleotide on an electrode microarray. The electrode microarray and preparation thereof was the same as for Example 1. The probe oligonucleotide was synthesized as in Example 1 and was also a 45-mer DNA unit. The electrodes were used as cathodes. The anode was an off array platinum anode. The dehybridizing solution was 1×PBS.

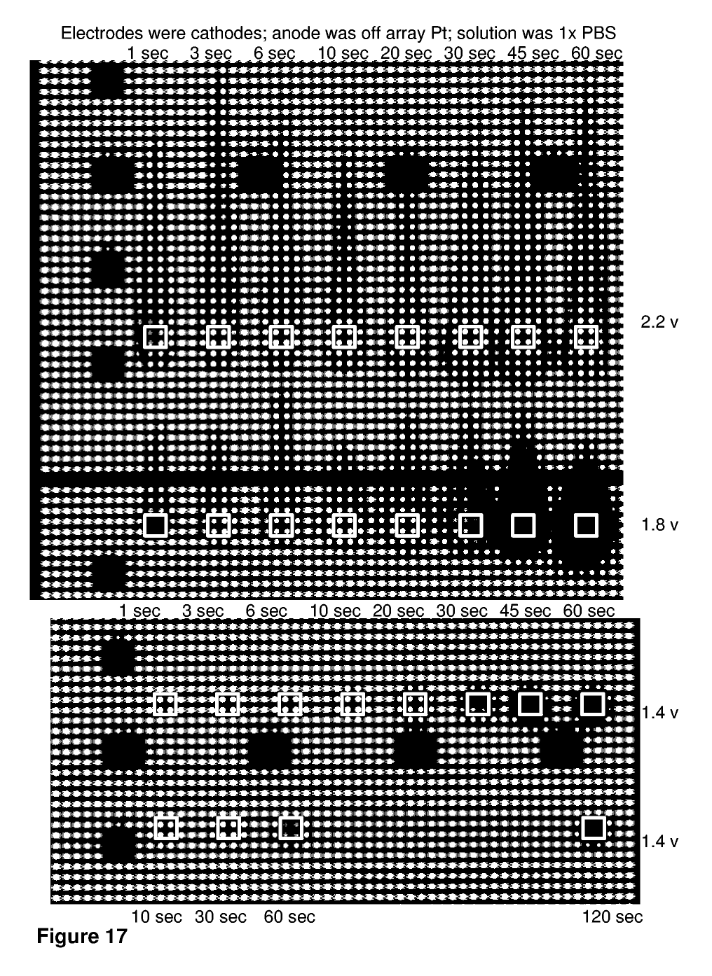

Referring to FIG. 17, the figure is a black and white conversion of a gray scale image of the electrode microarray after undergoing dehybridization. The time of the voltage pulse was varied as shown in the figure. The voltages used were 2.2, 1.8, and 1.4 volts as shown in the figure. The number of electrodes per pulse was kept constant at four electrodes as shown in the figure. The white squares were added to the original image of the microarray in order to identify the electrodes undergoing dehybridization (activation.)

The four by three and three by three electrode areas that are dark are electrodes that did not have probe oligonucleotide synthesized thereon. The small white horizontal striations coming off from each electrode fluorescent image are a result of saturation of the image taken by the scanner. Using an excess of target oligonucleotide caused the saturation in the image. The electrode microarray was in a vertical orientation during dehybridization. Loss of fluorescence indicates dehybridization.

As seen in FIG. 17, the loss of fluorescence increased as the time of the voltage pulse increased. Also, as the level of voltage increased, the loss of fluorescence increased. The higher brightness of the electrodes at the 2.2 voltage level is more of an artifact of the conversion to a black and white image. The darker areas around and above the four active electrodes indicate a loss of fluorescence. The darker areas increased with an increase in time of the voltage pulse. The loss of fluorescence shows that there was dehybridization at the active electrodes as well as at nearby non-active electrodes. The dehybridization at the non-active electrodes indicates pour containment of the ECG base.

### EXAMPLE 3

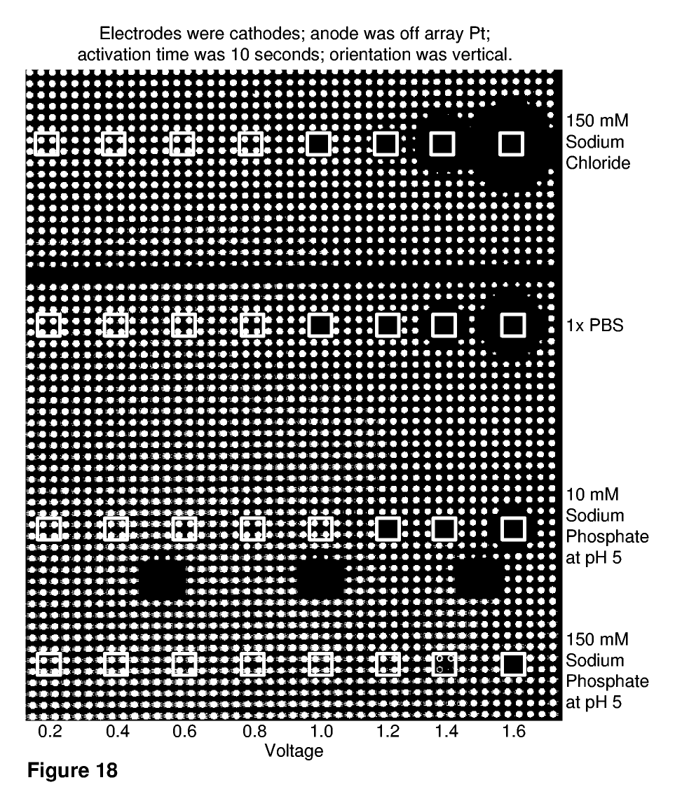

This example is identical to Example 2 except that (1) the voltage pulse was kept constant at 10 seconds, (2) the range of voltages increased and was from 0.2 volts to 1.6 volts, and (3) four different dehybridization solutions were used. The solutions used were 150 mM sodium chloride, 1×PBS, 10 mM sodium phosphate at pH 5, and 150 mM sodium phosphate at pH 5. The results are shown in FIG. 18. The same general comments in Example 2 about the electrode microarray image in FIG. 17 apply to FIG. 18 with respect to the electrode images.

FIG. 18 verifies that as voltage is increased, the loss of fluorescence increases and hence dehybridization increases. Most importantly, FIG. 18 shows that only the 150 mM sodium phosphate pH 5 solution confined the ECG base to the active electrodes at all voltage levels. At a 10 second pulse and a voltage of 1.6 volts, the dehybridization of the four electrodes in the 150 mM sodium phosphate pH 5 solution was complete and was confined to the active electrodes.

### EXAMPLE 4

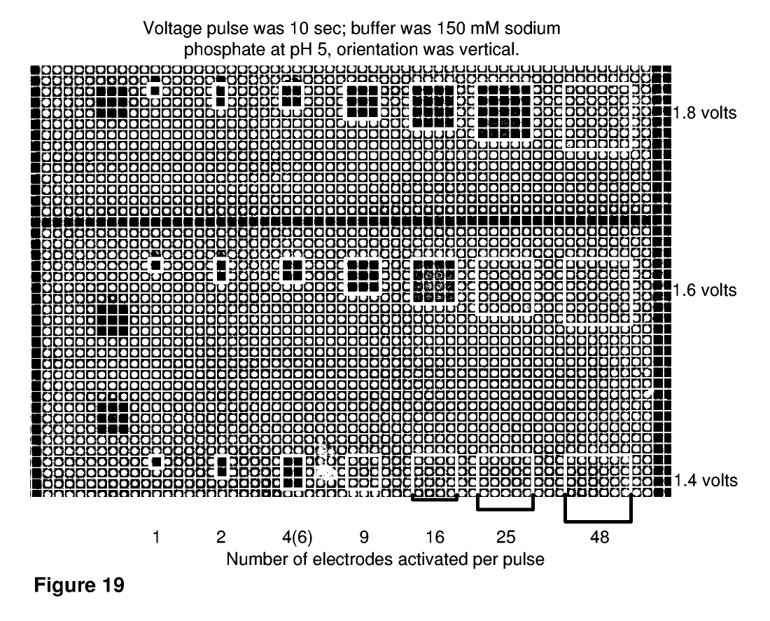

This example is the same as example 3 except that (1) the number of active electrodes was varied from 1 to 48 (1, 2, 4, 9, 16, 25, and 48,) (2) only the 150 mM sodium phosphate pH 5 dehybridization solution was used, and (3) the voltage was at only three different levels (1.8, 1.6, and 1.4.) Also, only 100 picomolar of target oligonucleotide was used in the hybridization solution; the lower concentration allowed visualizing any slight loss of fluorescence due to dehybridization. The results are shown in FIG. 19. The same general comments in Example 2 about the electrode microarray image in FIG. 17 apply to FIG. 17.

FIG. 19 shows that the 150 mM sodium phosphate pH 5 solution was fairly effective at confinement of the ECG base to the active electrodes as seen by the lack of spread of the loss of dehybridization. However, at the lower number of active electrodes (e.g, 1, 2, 4, and 6,) the electrodes above the active electrodes had a loss of fluorescence indicating dehybridization at those inactive electrodes. Thus, the vertical orientation of the electrode microarray appears to be preventing complete containment of the ECG base to the active electrodes.

Also noteworthy in FIG. 19 is that as the number of active electrodes increases in for a voltage pulse, the amount of dehybridization decreases for a given voltage. Thus, at 1.8 volts, the degree of dehybridization at 25 electrodes per pulse is good but is not as good at 48 active electrodes. At 1.6 volts, the loss of dehybridization appears to occur after 16 active electrodes. At 1.4 volts, the loss of dehybridization appears to occur after 6 active electrodes. The original gray scale image of the microarray provides that the loss of dehybridization is more gradual that indicated by the converted black and white image of FIG. 19.

### EXAMPLE 5

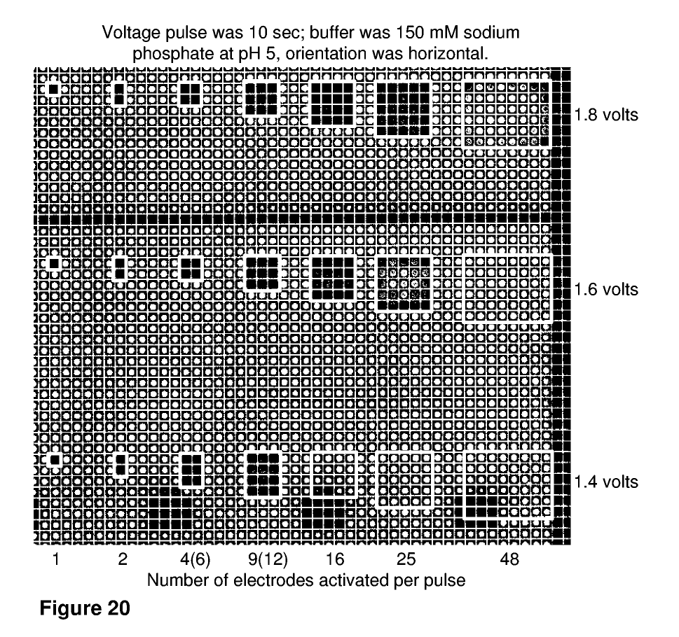

This example is essentially identical to Example 4 except that the electrode microarray was held in a horizontal orientation during dehybridization. Minor differences from Example 4 include (1) that at 1.4 volts and in one pulse, 12 electrodes were activated instead of 9 and (2) that there is an overlap between an area having no probe oligonucleotide and the area with 48 activated electrodes at 1.4 volts. The results are shown in FIG. 20. The general same comments in Example 2 about the electrode microarray image in FIG. 17 apply to FIG. 20. Most noteworthy of FIG. 20 is that the horizontal orientation of the electrode microarray has provided essentially complete containment of the ECG base to the active electrodes. As with FIG. 19 in Example 4, there is a loss of the degree of dehybridization as the number of active electrodes increases per voltage pulse.

### EXAMPLE 6

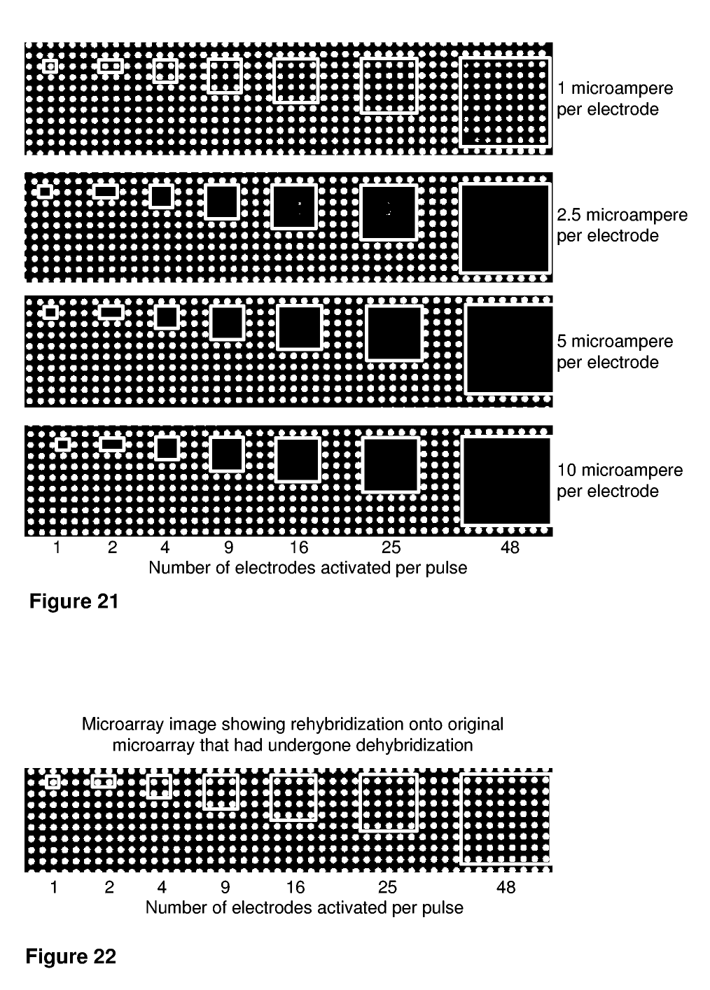

This example is the same as Example 5 except for the following differences. First, instead of using a constant voltage per pulse, a constant current was used. Four current levels were used: 1, 2.5, 5, or 10 microamperes per electrode. Second, the number of electrodes active was: 1, 2, 4, 9, 16, 25, or 48. Third, the concentration of target oligonucleotide was 3 nanomolar instead of 100 picomolar. The results are shown in FIG. 21. Again, the same general comments about the microarray image in FIG. 17 of Example 2 apply to FIG. 21, most notably, the loss of detail in the conversion to a black and white image from a grayscale image.

FIG. 21 provides that there is complete dehybridization at 5 and 10 microamperes per electrode. At 2.5 microamperes per electrode, there is still some lightness in the original image. At 1 microamperes per electrode, there is a small amount of dehybridization. The final voltage for the 10 microamperes per electrode experiment was 3.5 volts when 48 electrodes were activated. This example shows that complete dehybridization and confinement of the ECG base is attained when the dehybridization solution is 150 mM sodium phosphate pH 5, a current pulse is used, the activation time is 10 seconds, the pulse is approximately 5 microamperes per electrode or higher, the electrode microarray is held horizontal, and the electrodes are used as cathodes thus making ECG base.

### EXAMPLE 7

This is an example of a dehybridization experiment according to Example 6 followed by a rehybridization to the same microarray. For the current, the dehybridization step used 10 microampere per electrode. The purpose of this experiment was to verify that the ECG base was not removing the probe oligonucleotide in addition to the target oligonucleotide. The conditions of the rehybridization were the same as the initial hybridization of the Cy5 DIRECT® labeled 45-mer target oligonucleotide. The results are shown in FIG. 22, which is a black and white conversion of a gray scale image. The results show that upon rehybridization to the same microarray, the target oligonucleotide in the new solution rehybridized onto the electrodes where target oligonucleotide was previous removed by ECG base. Although there is some loss of fluorescence, the amount of loss is not sufficient to indicate significant removal of the probe oligonucleotide. Thus, the probe oligonucleotide is not substantially removed during dehybridization of the target oligonucleotide.

### EXAMPLE 8

This example shows that the presence of hydroquinone in an acidic solution affects the pH at which a double stranded deoxyribonucleic acid duplex will dehybridize into single stranded form. The duplex comprised target oligonucleotide and probe oligonucleotide. The probe oligonucleotide comprised KRAS 15-mer sequence. The target oligonucleotide comprised the complement of KRAS 15-mer having TEXAS RED® as a fluorescent label on the 3′ end. The microarray had the target oligonucleotide hybridized to probe oligonucleotide. The probe oligonucleotide was electrochemically synthesized in situ on the electrode microarray. Specifically, the probe oligonucleotide was a DNA unit. The electrode microarray was a prototype array similar to a CombiMatrix Corporation CUSTOMARRAY 902™, which has 1,000 electrodes in just over 1 cm square of area. Each electrode on the microarray had the same probe DNA having sequence 5′ tacgccaccagctcc [SEQ ID NO:2].

To synthesize the probe oligonucleotide, an electrode microarray was provided having a porous reaction layer on the electrodes. The electrodes were platinum. Standard phosphoramidite chemistry was used to synthesize the probe oligonucleotide coupled with electrochemical deblocking to remove the protecting group for the addition of each subsequent oligonucleotide unit (A, C, G, or T/U.) For each electrochemical-deblocking step, 1.8 volts were applied for 60 seconds. For a 1 liter solution, the deblocking solution comprised 100 ml methanol, 900 ml acetonitrile, 0.5 g anthraquinone, 5.5 g hydroquinone, and 15 g tetraethylammonium p-toluenesulfonate.

A hybridization solution comprised 5 nanomolar of target oligonucleotide in 2×PBS with 0.05% TWEEN® 20. The target oligonucleotide was the complement of the probe oligonucleotide. The solution was contacted to the electrode microarray to hybridize the target oligonucleotide to the probe oligonucleotide. The hybridization conditions comprised 37 degrees centigrade for 60 minutes. After hybridization, the microarray was viewed under a fluorescent imager to assess the degree of hybridization. The fluorescent imager used for this experiment was an Olympus BX60. Imaging showed hybridization of the probe to the target.

After hybridization, buffered solutions were made at different pH and with different amounts of hydroquinone. The hybridized microarray was exposed to the solutions to determine at what pH and hydroquinone concentration that dehybridization occurred. The buffered solutions comprised 50 millimolar citrate-phosphate. The pH of one solution not having hydroquinone was decreased by addition of hydrochloric acid until the pH reached 4, which is where dehybridization occurred.

The second solution had hydroquinone added to it at a concentration of 30% of the saturation value. The saturation value of hydroquinone in solution was not directly determined. Saturation was achieved by adding hydroquinone until no more would dissolve. To obtain a solution of less than saturation value, the saturated solution was mixed with solution having no hydroquinone according to volume, e.g., 30% saturated solution plus 70% solution without hydroquinone provides a 30% saturated solution. Hydrochloric acid was added to this solution until the pH dropped to 4.5, which is where dehybridization occurred.

The third solution had hydroquinone added to it at a concentration of 65% of the saturation value. Hydrochloric acid was added to this solution until the pH dropped to 5, which is where dehybridization occurred. For each solution, dehybridization was detected by fluorescent detection of TEXAS RED® label on the target oligonucleotide.

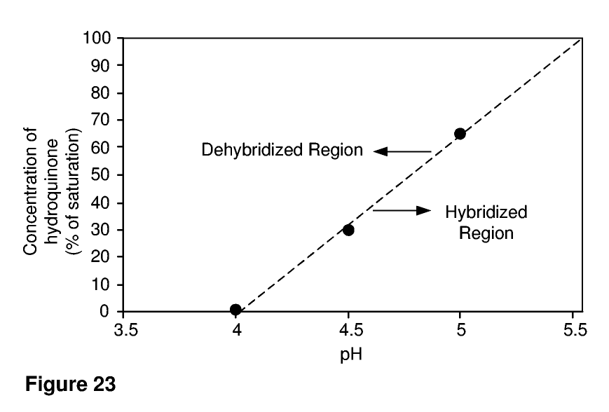

The results of these experiments are shown in FIG. 23. The figure shows the percentage of hydroquinone saturation versus the pH of the solution. Three data points are shown having a dotted line through the points. In the region to the left of the dotted line, there is dehybridization of oligonucleotide duplex. In the region to the right of the dotted line, the duplex remains intact. Extrapolating the line shows that a 100% saturated solution of hydroquinone should cause dehybridization at approximately a pH of 5.5. Thus, by adding hydroquinone to a dehybridization solution, the pH at which dehybridization occurs can be adjusted accordingly. Alternatively, an electrode microarray can be contacted to a dehybridization solution having hydroquinone while selected electrodes are activated as cathodes to prevent dehybridization at those electrodes while the inactive electrodes undergo dehybridization.

### EXAMPLE 9

This is an example of chemical dehybridization on an electrode microarray of a double stranded deoxyribonucleic acid duplex using solutions of different pH containing hydroquinone. The duplex comprised target oligonucleotide and probe oligonucleotide. The probe oligonucleotide comprised KRAS 15-mer. The target oligonucleotide comprised the complement of KRAS 15-mer having TEXAS RED® as a fluorescent label on the 3′ end.

The ECG base was used to prevent chemical dehybridization at selected electrodes that were activated as cathodes. The ECG base increased pH sufficiently to prevent chemical (pH) dehybridization. The microarray had the target oligonucleotide hybridized to probe oligonucleotide. The probe oligonucleotide was electrochemically synthesized in situ on the electrode microarray. Specifically, the probe oligonucleotide was a DNA unit. The electrode microarray was a CombiMatrix Corporation CUSTOMARRAY 902™. Each electrode on the microarray had the same probe DNA having sequence 5′ tacgccaccagctcc [SEQ ID NO:2].

To synthesize the probe oligonucleotide, an electrode microarray was provided having a porous reaction layer on the electrodes. The electrodes were platinum. Standard phosphoramidite chemistry was used to synthesize the probe oligonucleotide coupled with electrochemical deblocking to remove the protecting group for the addition of each subsequent oligonucleotide unit (A, C, G, or T/U.) For each electrochemical-deblocking step, 1.8 volts were applied for 60 seconds. For a 1 liter solution, the deblocking solution comprised 100 ml methanol, 900 ml acetonitrile, 0.5 g anthraquinone, 5.5 g hydroquinone, and 15 g tetraethylammonium p-toluenesulfonate.

A hybridization solution comprised 5 nanomolar of target oligonucleotide in 2×PBS with 0.05% TWEEN® 20. The target oligonucleotide was the complement of the probe oligonucleotide. The solution was contacted to the electrode microarray to hybridize the target oligonucleotide to the probe oligonucleotide. The hybridization conditions comprised 37 degrees centigrade for 60 minutes.

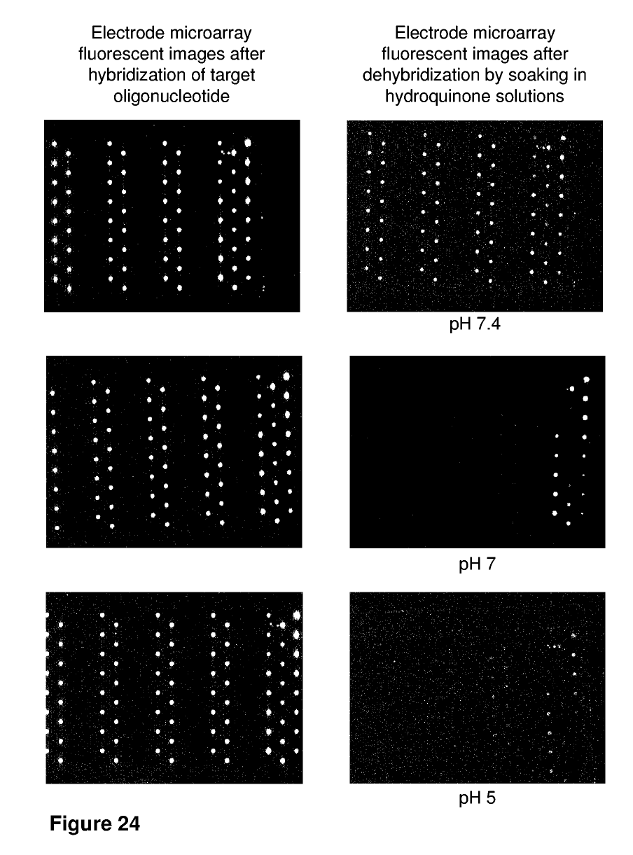

After hybridization, the microarray was viewed under a fluorescent imager to assess the degree of hybridization. The fluorescent imager used for this experiment was an Olympus BX60. FIG. 24 is a black and white conversion of an original gray scale image of the top view of a portion of an electrode microarray after hybridization of the target oligonucleotide to the probe oligonucleotide. The images on the left side of FIG. 24 are before chemical dehybridization. The images on the rights side are after chemical dehybridization using solutions at different pH. The pH 7.4 solution comprised 1×PBS saturated with hydroquinone. The pH 7.0 solution comprised 1×PBS saturated with hydroquinone and the pH was adjusted with HCl. The pH 5.0 solution comprised 50 millimolar citrate-phosphate buffer having 0.2 molar Na₂SO₄ saturated with hydroquinone. The exposure time to the chemical dehybridization solutions was 10 minutes.

The white spots of FIG. 24 represent the imaging of the TEXAS RED® on the target oligonucleotide and correspond to the electrodes having the probe oligonucleotide. As pH is decreased, the amount of fluorescence decreased, thus indicating that dehybridization is increasing. At pH 5.0, there is almost no fluorescence as would be predicted based on FIG. 23.

### EXAMPLE 10

This is an example of selective dehybridization on an electrode microarray of a double stranded deoxyribonucleic acid duplex using an acidic solution containing hydroquinone while making ECG base at selected electrodes to prevent dehybridization at those electrodes. An electrode microarray was prepared having target oligonucleotide hybridized to probe oligonucleotide according to Example 9.

The electrode microarray was placed in a chemical dehybridization solution while selected electrodes were maintained at −0.8 volts (cathodes.) The anode was an off-array Pt electrode. ECG base was made at the activated electrodes. The chemical dehybridization solution comprised 0.45 molar LiClO₄, 5 millimolar citrate-phosphate buffer, and 0.02 molar Na₂SO₄ and was saturated with hydroquinone. The pH was 5.0. The microarray was exposed to the solution for 10 minutes.

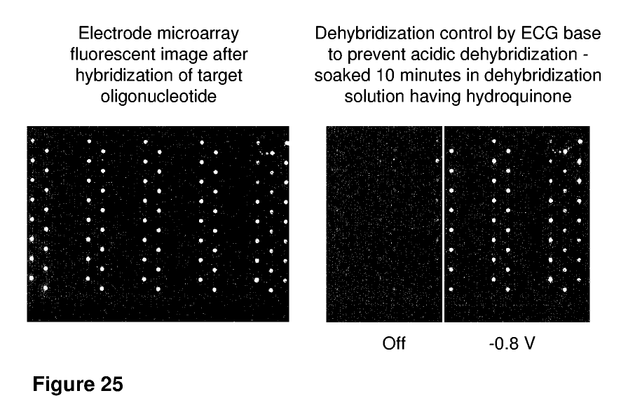

FIG. 25 provides the results. On the left side of the figure is a black and white image of the electrode microarray prior to chemical dehybridization. On the right side of the figure is a black and white image of the electrode microarray after chemical dehybridization while electrodes on the right side of the image were protected using ECG base. As can be seen, the ECG base was effective at preventing chemical dehybridization by raising pH at the active electrodes.

What is claimed is:

1. A method for selective dehybridization by electrochemically-generated reagent on an electrode microarray comprising: (a) providing an electrode microarray having an lectrode surface having at least one electrode proximate to a porous reaction layer having at least one oligonucleotide duplex, wherein the at least one oligonucleotide duplex comprises a target oligonucleotide and a probe oligonucleotide, wherein a dehybridizing solution contacts the porous reaction layer and the electrode surface; and (b) dehybridizing the at least one oligonucleotide duplex by generating an electrochemically-generated reagent, whereby the target oligonucleotide goes into the dehybridizing solution the probe nucleotide substantially remains attached to the porous reaction layer.

2. The method of claim 1 wherein the porous reaction layer is attached to the at least one electrode.

3. The method of claim 1 wherein the porous reaction layer is attached to an opposing surface to the electrode surface.

4. The method of claim 1 wherein the activation means comprises: application of a constant voltage to the at least one electrode of an absolute value of approximately 0.1 to 10.0 volts.

5. The method of claim 1 wherein the electrochemically-generated reagent is generated by a constant current applied to the at least one electrode having an absolute value of approximately 0.1 to 20 microampere per electrode.

6. The method of claim 1 wherein the dehybridization solution comprises a buffer having a concentration of approximately 1 to 1000 millimolar of buffer and a pH of about 5 to about 9.

7. The method of claim 11 wherein the buffer is selected from the group consisting of di-sodium phosphate, mono-sodium phosphate, citrate, carbonate, bicarbonate, borate, acetate, MES, Bis-Tris, ADA, ACES, PIPES, MOPSO, Bis-Tris Propane, BES, MOPS, HEPES, TES, DIPSO, TAPSO, TRIZMA, HEPPSO, POPSO, EPPS, TEA, Tricine, Bicine, TAPS, AMPSO, CHES, CAPSO, AMP, CAPS and combinations thereof.

8. A method for selective dehybridization by electrochemically-generated reagent on an electrode microarray comprising: (a) providing an electrode microarray having an electrode surface having at least a first electrode and a second electrode, wherein the first electrode is proximate to a first porous reaction layer and the second electrode is proximate to a second porous reaction layer; (b) binding a first probe oligonucleotide to the first porous reaction layer and a second probe oligonucleotide to the second porous reaction layer; (c) hybridizing a first target oligonucleotide to the first probe oligonucleotide and a second target oligonucleotide to the second probe oligonucleotide, wherein the first target oligonucleotide and the first probe oligonucleotide form a first oligonucleotide duplex and the second target oligonucleotide and the second probe oligonucleotide form a second oligonucleotide duplex; and (d) dehybridizing the first oligonucleotide duplex by an electrochemically-generated reagent generated in a dehybridizing solution contacting the first porous reaction layer, the second reaction layer, and the electrode surface, wherein the electrochemically-generated reagent is generated by an activation means applied to the first electrode, whereby the first target nucleotide goes into the dehybridizing solution, the first probe oligonucleotide substantially remains attached to the first reaction layer, and the second oligonucleotide duplex is not dehybridized.

9. The method of claim 8 wherein the first porous reaction layer is attached to the first electrode and the second porous reaction layer is attached to the second electrode.

10. The method of claim 8 wherein the first porous reaction layer is attached to an opposing surface to the electrode surface and the second porous reaction layer is attached to an opposing surface to the electrode surface.

11. The method of claim 8 wherein the activation means comprises: application of a constant voltage to the at least one electrode of an absolute value of approximately 0.1 to 10.0 volts.

12. The method of claim 8 wherein the activation means comprises: application of a constant current to the first electrode of an absolute value of approximately 0.1 to 20 microampere per electrode.

13. The method of claim 8 wherein the dehybridization solution comprises a buffer having a concentration of approximately 1 to 1000 millimolar of buffer and a pH of approximately 5 to 9.

14. The method of claim 13 wherein the buffer is selected from the group consisting of di-sodium phosphate, mono-sodium phosphate, citrate, carbonate, bicarbonate, borate, acetate, MES, Bis-Tris, ADA, ACES, PIPES, MOPSO, Bis-Tris Propane, BES, MOPS, HEPES, TES, DIPSO, TAPSO, TRIZMA, HEPPSO, POPSO, EPPS, TEA, Tricine, Bicine, TAPS, AMPSO, CHES, CAPSO, AMP, CAPS and combinations thereof.

15. A method for selective dehybridization by electrochemically-generated reagent on an electrode microarray comprising: (a) providing an electrode microarray having an electrode surface having at least a first electrode and a second electrode, wherein the first electrode is proximate to a first porous reaction layer having a plurality of first oligonucleotide duplexes and the second electrode is proximate to a second porous reaction layer having a plurality of second oligonucleotide duplexes, wherein each of the plurality of first oligonucleotide duplexes comprise a first probe oligonucleotide and a first target oligonucleotide and each of the plurality of second oligonucleotide duplexes comprise a second probe oligonucleotide and a second target oligonucleotide; and (b) applying a dehybridization solution to the electrode microarray; and (c) generating an electrochemical reagent at the second electrode, whereby the electrochemically-generated reagent prevents dehybridization of the plurality of second oligonucleotide duplexes.

16. The method of claim 15 wherein the first porous reaction layer is attached to the first electrode and the second porous reaction layer is attached to the second electrode.

17. The method of claim 15 wherein the first porous reaction layer is attached to an opposing surface to the electrode surface and the second porous reaction layer is attached to an opposing surface to the electrode surface.

18. The method of claim 15 wherein the first probe oligonucleotide and the second probe oligonucleotide are synthesized in situ by an electrochemical synthesis means.

19. The method of claim 15 wherein the first probe oligonucleotide and the second probe oligonucleotide are presynthesized and attached to the reaction layer.

20. The method of claim 15 wherein generating the electrochemical reagent comprises either (a) applying a constant voltage to at least one electrode having an absolute value of approximately 0.1 to 10.0 volts or (b) applying a constant current to at least one electrode having an absolute value of approximately 0.1 to 20 microampere per electrode.

21. The method of claim 15 wherein the chemical dehybridization solution comprises a solution selected from the group consisting of (a) an aqueous solution of buffer from approximately 1 to 1000 millimolar; (b) an organic buffer that modifies the pH of dehybridization solution at a concentration from approximately 1 to 100% of the saturation value of the organic buffer; and (c) a pH modifying substance in an amount sufficient to adjust the pH to a value of approximately below 5.5 or above approximately 10.0.

22. The method of claim 21 wherein the aqueous buffer is selected from the group consisting of di-sodium phosphate, mono-sodium phosphate, citrate, carbonate, bicarbonate, borate, acetate, MES, Bis-Tris, ADA, ACES, PIPES, MOPSO, Bis-Tris Propane, BES, MOPS, HEPES, TES, DIPSO, TAPSO, TRIZMA, HEPPSO, POPSO, EPPS, TEA, Tricine, Bicine, TAPS, AMPSO, CHES, CAPSO, AMP, CAPS and combinations thereof.

23. The method of claim 21 wherein the organic buffer is selected from the group consisting of hydroquinone, catechol, p-aminophenol, o-pnenylenediamine, p-pnenylenediamine, and combinations thereof.

24. A method for selective dehybridization by electrochemically-generated reagent on an electrode microarray comprising: (a) providing an electrode microarray having an electrode surface having at least a first electrode and a second electrode, wherein the first electrode is proximate to a first porous reaction layer and the second electrode is proximate to a second porous reaction layer; (b) binding a first probe oligonucleotide to the first porous reaction layer and a second probe oligonucleotide to the second porous reaction layer; (c) hybridizing a first target oligonucleotide to the first probe oligonucleotide and a second target oligonucleotide to the second probe oligonucleotide, wherein the first target oligonucleotide and the first probe oligonucleotide form a first oligonucleotide duplex and the second target oligonucleotide and the second probe oligonucleotide form a second oligonucleotide duplex; (d) applying a chemical dehybridizing solution to the electrode microarray; and (e) generating an electrochemically-generated reagent at the second electrode and second porous reaction layer, whereby the electrochemically-generated reagent prevents dehybridization of the plurality of second oligonucleotide duplexes.

25. The method of claim 24 wherein the first porous reaction layer is attached to the first electrode and the second porous reaction layer is attached to the second electrode.

26. The method of claim 24 wherein the first porous reaction layer is attached to an opposing surface to the electrode surface and the second porous reaction layer is attached to an opposing surface to the electrode surface.

27. The method of claim 24 wherein generating the electrochemical reagent comprises either (a) applying a constant voltage to at least one electrode having an absolute value of approximately 0.1 to 10.0 volts or (b) applying a constant current to at least one electrode having an absolute value of approximately 0.1 to 20 microampere per electrode.

28. The method of claim 24 wherein the chemical dehybridization solution comprises a solution selected from the group consisting of (a) an aqueous solution of buffer from approximately 1 to 1000 millimolar; (b) an organic buffer that modifies the pH of dehybridization solution at a concentration from approximately 1 to 100% of the saturation value of the organic buffer; and (c) a pH modifying substance in an amount sufficient to adjust the pH to a value of approximately below 5.5 or above approximately 10.0.


SEQUENCE LISTING (2 sequences)

SEQ ID NO: 1 - DNA, 45 bp, Artificial Sequence
  An artificial 45-mer DNA synthesized in situ and used for complex hybridizations.
cataacgatggtggcgatgttaacctcggcttatggcgtcaccgg

SEQ ID NO: 2 - DNA, 15 bp, Homo Sapien
tacgccaccagctcc